(19) United States
(12) Patent Application Publication
(10) Pub. No.: US 2026/0106039 A1
(43) Pub. Date: Apr. 16, 2026

(54) SYSTEMS AND METHODS FOR HEALTH STATUS VERIFICATION AND SECURE DATA SHARING

(71) Applicant: Plum Check, Inc., New York, NY (US)

(72) Inventors: Joshua Karetny, New York, NY (US); Celine Gounder, New York, NY (US)

(73) Assignee: Plum Check, Inc., New York, NY (US)

(21) Appl. No.: 19/318,141

(22) Filed: Sep. 3, 2025

Related U.S. Application Data
(60) Provisional application No. 63/707,047, filed on Oct. 14, 2024.

(51) Int. Cl. G16H 50/30; G06F 21/31; G16H 10/60

(52) U.S. Cl. CPC G16H 50/30; G06F 21/31; G16H 10/60

(57) ABSTRACT

A provider computing system includes a processing circuit configured to receive personal data of a user, create a user account for the user, receive identity verification information of the user via a first application programming interface (API), provide a request for health data of the user to a medical record provider computing system via a second API, receive the health data of the user via the second API, use a logic-driven translation layer to determine a health status of the user based on the health data, and facilitate API-based sharing of the health status leading to display on a third-party user device of a third party via a client application, via a platform computing system, or via a user device of the user.

### CROSS-REFERENCE TO RELATED APPLICATION(S)

This application claims the benefit of and priority to U.S. Provisional Patent Application No. 63/707,047, filed Oct. 14, 2024, the entire disclosure of which is incorporated by referenced herein in its entirety and for all purposes.

### BACKGROUND

With the rise of electronic health records (EHRs) and digital platforms, consumers increasingly seek to share aspects of their health data electronically in usable formats. However, EHRs often store data using codes and values that would be incomprehensible except to the trained health care professional. In addition, the EHRs may lack the context to explain the relevance of the information included in the record or the significance of information not included. Consequently, consumers lack secure and verified ways to share health information with others and may resort to sharing health information over the course of conversation, sending or showing screen shots of test reports and other health information, or emailing files that may be over-inclusive and intrusive given the context. These ad-hoc, unverified methods of exchanging health information are inadequate and rely on files and images that can be manufactured and do not protect the sensitive data from being forwarded to others by the recipient. Furthermore, digital platforms, including social media and other consumer facing applications, often do not verify the identities of their users further frustrating the exchange of health information.

### SUMMARY

One embodiment relates to a provider computing system. The provider computing system includes a processing circuit configured to receive personal data of a user, and create a user account associated with the provider computing system for the user based on the personal data of the user. The processing circuit is further configured to receive, via a first application programming interface (API), identity verification information of the user from an identity verification computing system. The processing circuit is further configured to provide, via a second API, a request for health data of the user to a medical record provider computing system. The processing circuit is further configured to receive, via the second API, the health data of the user from the medical record provider computing system. The processing circuit is further configured to determine a health status of the user based on the health data, and cause the health status of the user to be displayed on a third-party user device of a third party via a client application or via a platform computing system.

Another embodiment relates to a provider computing system. The provider computing system includes a processing circuit configured to receive personal data of a user. The processing circuit is further configured to provide, via an application programming interface (API), a request for health data of the user to a medical record provider computing system. The processing circuit is further configured to receive, via the API, health data of the user from the medical record provider computing system. The processing circuit is further configured to determine a health status of the user based on the health data, and cause the health status of the user to be displayed on at least one of a user device associated with the user via a first instance of a client application, a third-party user device of a third party via a second instance of the client application, or via a platform computing system.

Another embodiment relates to a method. The method includes receiving, by a processing circuit of a provider computing system, personal data of a user. The method further includes providing, by the processing circuit and via an application programming interface (API), a request for health data of the user to a medical record provider computing system. The method further includes receiving, by the processing circuit and via the API, health data of the user from the medical record provider computing system. The method further includes determining, by the processing circuit, a health status of the user based on the health data. The method further includes causing, by the processing circuit, the health status of the user to be displayed on at least one of a user device associated with the user via a first instance of a client application, a third-party user device of a third party via a second instance of the client application, or via a platform computing system.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of a system for providing an identity verification and health data interpretation service, according to an example embodiment.

FIG. 2 is a block diagram of the provider computing system of FIG. 1, according to an example embodiment.

FIG. 3 is a block diagram of a medical record provider computing system of FIG. 1, according to an example embodiment.

FIG. 4 is a block diagram of an identity verification computing system of FIG. 1, according to an example embodiment.

FIG. 5 is a block diagram of a platform computing system of FIG. 1, according to an example embodiment.

FIG. 6 is a block diagram of a user device of FIG. 1, according to an example embodiment.

FIG. 7 is a flow chart illustrating a method for providing a verified identity and health status of a user, according to an example embodiment.

FIG. 8 is a flow chart illustrating a method for determining a sexually transmitted infection (STI) health status of a user, according to an example embodiment.

FIG. 9 is a flow chart illustrating a method for determining a human immunodeficiency virus (HIV) health status of a user, according to an example embodiment.

FIG. 10 is a flow chart illustrating a method for determining a pre-exposure prophylaxis (PrEP) health status of a user, according to an example embodiment.

FIGS. 11A-11B is a flow chart illustrating a method for determining a vaccination health status of a user, according to an example embodiment.

FIG. 12 is a graphical user interface showing the user's profile, according to an example embodiment.

FIG. 13 is another graphical user interface showing the user's profile, according to an example embodiment.

FIG. 14 is another graphical user interface showing the user's profile, according to an example embodiment.

FIG. 15 is another graphical user interface showing the user's profile, according to an example embodiment.

FIG. 16 is a graphical user interface showing the user's profile for limited time period, according to an example embodiment.

FIG. 17 is another graphical user interface showing the user's profile for limited time period, according to an example embodiment.

FIG. 18 is another graphical user interface showing the user's profile, according to an example embodiment.

FIG. 19 is another graphical user interface showing the user's profile, according to an example embodiment.

FIG. 20 is another graphical user interface showing the user's profile for limited time period, according to an example embodiment.

FIG. 21 is another graphical user interface showing the user's profile for limited time period, according to an example embodiment.

### DETAILED DESCRIPTION

Referring generally to the figures, systems and methods for providing at least one of an identity verification or health data interpretation service are disclosed. The systems and methods disclosed herein are used to provide verified identity information and health status information to users of the system in a consistent format. For example, the systems and methods disclosed herein can be used to provide users on any type of digital platform verification of the user's identity and health status check information (e.g., vaccination status, medication status, etc.). As such, the systems and methods disclosed herein can be used to provide identity verification and health status check information of a customer to airline travel platforms, education platforms, child care platforms, elder care platforms, pet-sitting platforms, life insurance platforms, workplace/employer platforms, telehealth and e-prescribing platforms, heath record wallet platforms, dating platforms, and so on. In one example, the systems and methods disclosed herein may be used to provide users on a social media platform verification of a user's identity and information regarding a sexual wellness (e.g., whether a user has tested negative for a sexually-transmitted disease) or vaccination status (e.g., whether a user has received the COVID vaccine) of the user using consistent and clear graphic user interface icons. It will be appreciated that the systems and methods disclosed herein can be applicable to and used within any platform that requires an identity verification or health status check of a user using the platform for any purpose (e.g., booking travel, hiring someone or obtaining a job, receiving medical care, enrollment in an activity such as school or camp, or for receiving medical care).

The provider system, which is configured to provide identity verification and health status verification on a digital platform, offers several technical advantages. One benefit is the improvement in health data accuracy and reliability. Unlike current practices that rely on self-reported health information, which can be outdated or inaccurate, this system integrates directly with electronic health record (EHR) computing systems, which allows for real-time or consistently scheduled updates to users' health status, ensuring that the information provided is current and precise. The automatic updating of health data using graphical user interface icons also reduces the need for manual input of such information, or manually providing such information via individualized messages to other users, which reduces the processing load on the system and network bandwidth required by the system by eliminating the need for such tasks.

The system can ingest raw or semi-structured health data received via an application programming interface, translating the received health data via logic formulas, and converting the health data to consumer readable statuses (e.g., such as generated display icons). The system thereby transforms clinical health records into verified, human-readable health statuses for secure sharing via digital platforms.

Another advantage provided by the provider system is standardization of health data and compliance. By utilizing well-defined standards, the provider system not only ensures consistent health data reporting but also simplifies the process of integrating with other systems. This is particularly important in maintaining regulatory compliance and streamlining the addition of new services or integrations. Additionally, the provider system supports the creation of comprehensive health profiles, using a consistent form (e.g., text, badges, or other graphical user interface icons) to convey key health metrics such as test results, vaccinations, and prescribed medication data. This structured approach provides a more reliable framework than the ad-hoc solutions commonly used in current systems.

With respect to social media and dating platforms, the provider system also appeals to health-conscious users who might otherwise hesitate to engage with these platforms due to concerns about unreliable identity information or unreliable health information. As such, the provider system provides services that could not only expand the user base of such systems but also increase advertising revenue by providing identity and health data to users within the application, rather than user's seeking verification on third-party platforms.

The provider system's ability to aggregate and anonymize user data may also provide valuable insights into public health trends, which could drive strategic partnerships or services in the healthcare space.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

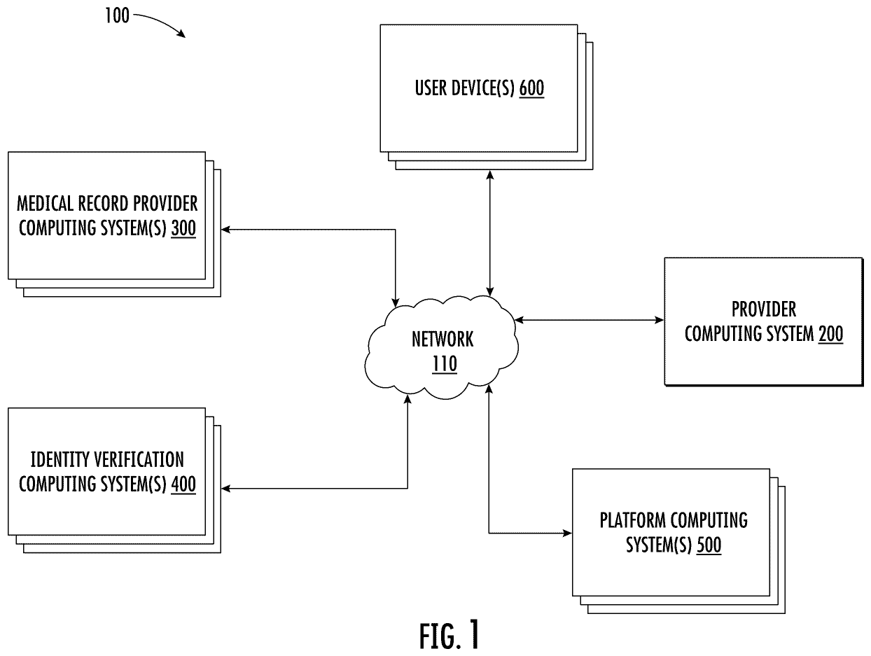

Referring to FIG. 1, a block diagram of a system 100 for providing an identity and health data interpretation service is shown according to an example embodiment. The system 100 includes a network 110, a provider computing system 200, one or more medical record provider computing systems 300, one or more identity verification computing systems 400, one or more platform computing systems 500, and one or more user devices 600. While for ease of reference each of the one or more medical record provider computing systems 300, one or more identity verification computing systems 400, one or more platform computing systems 500, and one or more user devices 600 may be described in the singular, it will be appreciated that any use of such terms in the singular can also mean the same term but in the plural.

The network 110 can be any system configured to enable communications between separate systems or devices that are otherwise remote from one another. The network 110 can include one or more of the Internet, a cellular network, Wi-Fi, Wi-Max, a proprietary communication network, a proprietary retail or service provider network, or other type of wired or wireless network. While only a single network 110 is shown, it will be appreciated that the systems and devices shown in FIG. 1 can communicate over any number of networks. For example, in some embodiments, the provider computing system 200 can be configured to communicate with the platform computing system 500 over a proprietary network, and be configured to communicate with the medical record provider computing system 300 and the identity verification computing systems 400 over the Internet.

The provider computing system 200, the medical record provider computing systems 300, the identity verification computing systems 400, the platform computing systems 500, and the user devices 600 can each be communicably coupled with one another via the network 110 and therefore configured to send data to and receive data from one another over the network 110. However, in some embodiments, the various systems and devices illustrated in FIG. 1 may be configured to have more limited communications capabilities such that one system may only be configured or authorized to communicate with certain other systems but not each of the systems and devices shown in FIG. 1. For example, in some embodiments, the medical record provider computing systems 300 and the identity verification computing systems 400 can be configured to communicate with the provider computing system 200 but not the other systems and devices illustrated in FIG. 1. In another example, in some embodiments, the platform computing system 500 can be configured to communicate with the provider computing system 200 and the user devices 600 but not the other systems illustrated in FIG. 1.

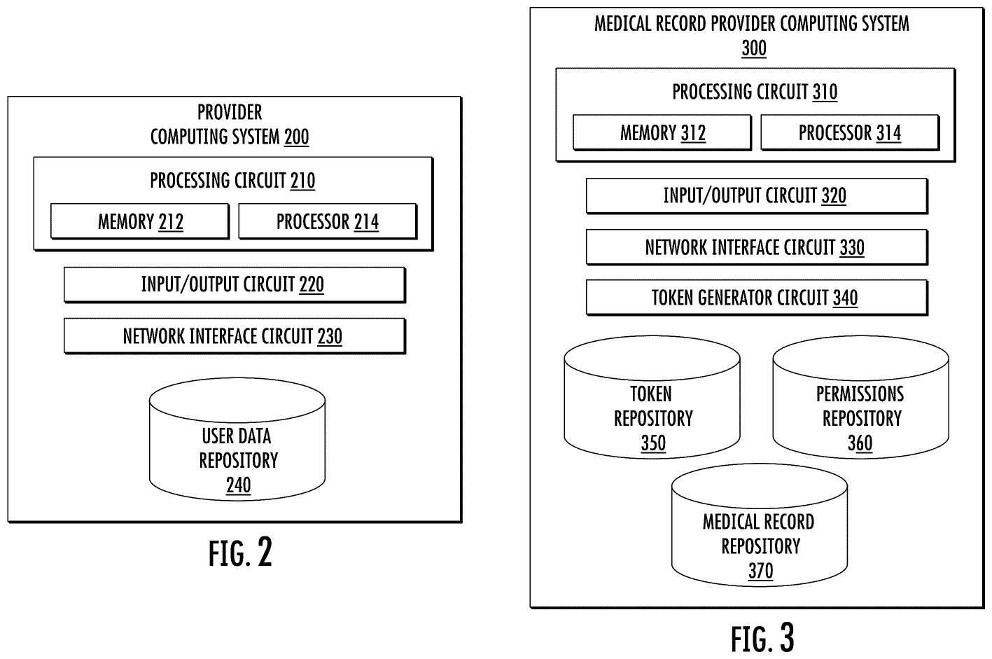

Referring to FIG. 2, a block diagram of the provider computing system 200 of FIG. 1 is shown according to an example embodiment. While referred to as the provider computing system 200, it will be appreciated that the provider computing system 200 can be configured as a standalone identity check computing system, a standalone health check computing system, or any combination of an identity check computing system, a health check computing system, and other types of computing system. For example, in some embodiments, the provider computing system 200 is an identity verification and health check computing system. In some embodiments, the provider computing system 200 is configured to provide one or more of verification of an identity, health status information, confirmation of student immunization records, a fitness-to-work assessment, information regarding health-related travel accommodations, or other health status information.

The provider computing system 200 includes a processing circuit 210, an input/output circuit 220, a network interface circuit 230, and a user data repository 240. The provider computing system 200 is configured to receive user inputs from the user device 600, receive health data from the medical record provider computing system 300, receive identity data from the identity verification computing system 400, interpret the received data, and provide health check data and identity check data to the platform computing system 500.

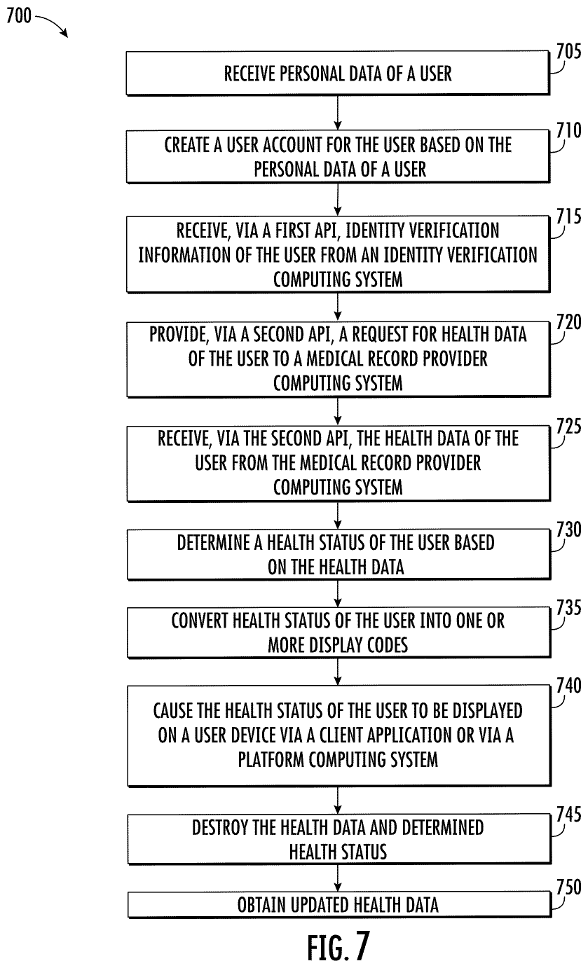

The processing circuit 210 includes memory 212 and processor 214. The processing circuit 210 can be configured to perform all functionalities of the provider computing system 200 as described herein, including the steps performed with reference to FIGS. 7-11.

The memory 212 may be one or more devices (e.g., RAM, ROM, Flash memory, hard disk storage) for storing data and/or computer code for completing and/or facilitating the various processes described herein. Memory 212 may be or include non-transient volatile memory, non-volatile memory, and non-transitory computer storage media. Memory 212 may include database components, object code components, script components, or other types of information structured for supporting the various activities and information structures described herein. The memory 212 may be coupled to the processor 214 and include computer code or instructions for executing one or more processes described herein.

The processor 214 may be implemented as one or more server processors, application specific integrated circuits (ASIC), field programmable gate arrays (FPGAs), digital signal processor (DSP), microprocessors, or other suitable electronic processing components. The server(s) or server computer may be geographically dispersed relative to other server(s) of the provider computing system 200. Further, there may be a variety of different types of server(s) included in the provider computing system 200 (e.g., application server, database server, catalog sever, virtual private network (VPN) server, communications server, web server, and so on). The memory device may be included with the server(s). The provider computing system 200 is configured to run a variety of application programs and store associated data in a database of the memory 212.

The input/output circuit 220 is configured to exchange data, communications, instructions, etc. with an input/output component of the provider computing system 200 (e.g., a keyboard, a mouse, etc.) (e.g., with an employee, non-employee, operator, etc. associated with the provider computing system 200). In some embodiments, the input/output circuit 220 is incorporated into an input/output device. For example, a laptop, desktop, or tablet computer may include the input/output circuit 220 such that the laptop, desktop, or tablet computer is communicably coupled to the provider computing system 200. The input/output circuit 220 is configured to receive communications from, and provide communications to, various employees, agents, or operators associated with the provider computing system 200.

The network interface circuit 230 is configured to establish communicable connections with other computing systems (e.g., the medical record provider computing systems 300, the identity verification computing systems 400, the platform computing systems 500, the user devices 600, and other computing systems), by way of the network 110. The network interface circuit 230 may include program logic that facilitates connection of the provider computing system 200 to the network 110. For example, the network interface circuit 230 may include a combination of a wireless network transceivers (e.g., a NFC transceiver, a Bluetooth transceiver, a Wi-Fi transceiver, etc.) and/or a wired network transceiver (e.g., an Ethernet transceiver). In some arrangements, the network interface circuit 230 includes the hardware and machine-readable media sufficient to support communication over multiple channels of data communication. Further, in some arrangements, the network interface circuit 230 includes cryptography capabilities to establish a secure or relatively secure communication session in which data communicated over the session is encrypted.

The user data repository 240 is configured to retrievably hold (e.g., in cache memory), store (e.g., in non-transitory memory), categorize, and/or otherwise serve as a repository for information pertaining to users of the provider computing system 200. For example, the user data repository 240 can include a name of the user, an age of the user, a home address of the user, a residence city of the user, a residence state of the user, a residence country of the user, an email address of the user, a telephone number of the user, API access tokens pertaining to the user, and display codes associated with the user based on the health status of the user determined from health data received from the medical record provider computing systems 300 and identity verification data received from the identity verification computing systems 400. In some embodiments, the user data repository 240 can be configured to store health data of the user or health status data of the user either temporarily or for long-term durations, though the provider computing system 200 can also destroy the health data and health status data of the user after determining and storing the display codes for the user. In some embodiments, the provider computing system 200 can destroy the health data and health status data of the user after a predetermined time period. For example, the provider computing system 200 can destroy the health data and health status data of the user anytime within the range of 1 day to 1 year. For example, the provider computing system 200 can destroy the health data and health status data of the user after 30 days, after 60 days, after 90 days, etc.

Referring to FIG. 3, a block diagram of a medical record provider computing system 300 of FIG. 1 is shown according to an example embodiment. The medical record provider computing system 300 includes a processing circuit 310, an input/output circuit 320, a network interface circuit 330, a token generator circuit 340, a token repository 350, a permissions repository 360, and a medical record repository 370. In an alternate embodiment, the token generator circuit 340, the token repository 350, and/or the permissions repository 360 are part of another computing system, accessed as needed by the medical record provider computing system 300 or the provider computing system 200.

The processing circuit 310 includes memory 312 and processor 314. The processing circuit 310 can be configured to perform all functionalities of the medical record provider computing system 300 as described herein.

The memory 312 may be one or more devices (e.g., RAM, ROM, Flash memory, hard disk storage) for storing data and/or computer code for completing and/or facilitating the various processes described herein. Memory 312 may be or include non-transient volatile memory, non-volatile memory, and non-transitory computer storage media. Memory 312 may include database components, object code components, script components, or other types of information configured for supporting the various activities and information structures described herein. The memory 312 may be coupled to the processor 314 and include computer code or instructions for executing one or more processes described herein.

The processor 314 may be implemented as one or more server processors, application specific integrated circuits (ASIC), field programmable gate arrays (FPGAs), digital signal processor (DSP), microprocessors, or other suitable electronic processing components. The server(s) or server computer may be geographically dispersed relative to other server(s) of the medical record provider computing system 300. Further, there may be a variety of different types of server(s) included in the medical record provider computing system 300 (e.g., application server, database server, catalog sever, virtual private network (VPN) server, communications server, web server, and so on). The memory device may be included with the server(s). The medical record provider computing system 300 is configured to run a variety of application programs and store associated data in a database of the memory 312.

The input/output circuit 320 is configured to exchange data, communications, instructions, etc. with an input/output component of the medical record provider computing system 300. In some embodiments, the input/output circuit 320 is incorporated into an input/output device. For example, a laptop, desktop, or tablet computer may include the input/output circuit 320 such that the laptop, desktop, or tablet computer is communicably coupled to the medical record provider computing system 300. The input/output circuit 320 is configured to receive communications from, and provide communications to, various employees, agents, or operators associated with the medical record provider computing system 300.

The network interface circuit 330 is configured to establish communicable connections with other computing systems (e.g., provider computing system 200, the identity verification computing systems 400, the platform computing systems 500, the user devices 600, and other computing systems), by way of the network 110. The network interface circuit 330 may include program logic that facilitates connection of the medical record provider computing systems 300 to the network 110. For example, the network interface circuit 330 may include a combination of a wireless network transceivers (e.g., a NFC transceiver, a Bluetooth transceiver, a Wi-Fi transceiver, etc.) and/or a wired network transceiver (e.g., an Ethernet transceiver). In some arrangements, the network interface circuit 330 includes the hardware and machine-readable media sufficient to support communication over multiple channels of data communication. Further, in some arrangements, the network interface circuit 330 includes cryptography capabilities to establish a secure or relatively secure communication session in which data communicated over the session is encrypted.

The token generator circuit 340 is configured to generate and/or otherwise create access tokens to be used in a token-based authentication to allow the provider computing system 200 to access an application programming interface (API) of the medical record provider computing system 300. Furthermore, the token generator circuit 340 is configured to generate access tokens which associatively map to both an expiration (e.g., a lifespan for the access token contained in the token repository 350) and a set of permissions stored in the permissions repository 360.

The token repository 350 is configured to retrievably hold (e.g., in cache memory), store (e.g., in non-transitory memory), categorize, and/or otherwise serve as a repository for information pertaining to access tokens, configuration options associated with the access tokens (e.g., an expiration), systems authorized to access information of the medical record provider computing system 300 (e.g., the provider computing system 200), and API tokens provisioned to systems authorized to access information of the medical record provider computing system 300 (e.g., the provider computing system 200). Accordingly, the token repository 350 is configured to retrievably store and access information pertaining to access rights of the provider computing system 200 (e.g., defining the specific health data the provider computing system 200 is authorized to receive for a particular user).

The permissions repository 360 is configured to retrievably hold (e.g., in cache memory), store (e.g., in non-transitory memory), categorize, and/or otherwise serve as a repository for information pertaining to access control rules governing what systems (e.g., the provider computing system 200) can interact with specific API resources of the medical record provider computing system 300, and what actions the systems are permitted to perform (e.g., what data the provider computing system 200 is authorized to receive from the medical record provider computing system 300).

The medical record repository 370 is configured to retrievably hold (e.g., in cache memory), store (e.g., in non-transitory memory), categorize, and/or otherwise serve as a repository for health data of users. For example, the medical record repository 370 can include a wide range of health-related data encompassing both clinical and administrative information, including user demographic details (e.g., names, addresses, and contact information), medical histories (e.g., past diagnoses, surgeries, allergies, family medical backgrounds), lab results, imaging reports, vital signs (e.g., blood pressure, heart rate), medication records detailing current and past prescriptions, treatment plans, progress notes from healthcare professionals, immunization records, procedures performed, ongoing monitoring data from devices or tests, billing information, and insurance details. In some embodiments, the health data stored by the medical record repository 370 includes medical codes, such as codes in using the Logical Observation Identifiers Names and Codes (LOINC) format, the Systematized Nomenclature of Medicine Clinical Terms (SNOMED CT) codes, or RxNorm though it will be appreciated that the health data can be received in any format.

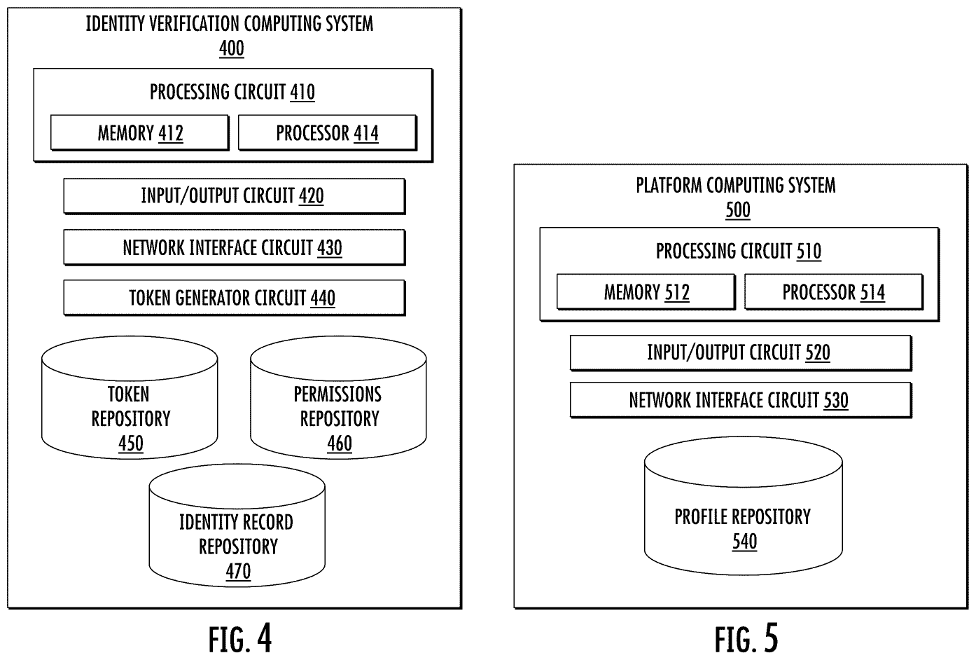

Referring to FIG. 4, a block diagram of an identity verification computing system 400 of FIG. 1 is shown according to an example embodiment. The identity verification computing system 400 includes a processing circuit 410, an input/output circuit 420, a network interface circuit 430, a token generator circuit 440, a token repository 450, a permissions repository 460, and an identity record repository 370. In an alternate embodiment, the token generator circuit 440, the token repository 450, and/or the permissions repository 460 are part of another computing system, accessed as needed by the identity verification computing system 400 or the provider computing system 200.

The processing circuit 410 includes memory 412 and processor 414. The processing circuit 410 can be configured to perform all functionalities of the identity verification computing system 400 as described herein.

The memory 412 may be one or more devices (e.g., RAM, ROM, Flash memory, hard disk storage) for storing data and/or computer code for completing and/or facilitating the various processes described herein. Memory 412 may be or include non-transient volatile memory, non-volatile memory, and non-transitory computer storage media. Memory 412 may include database components, object code components, script components, or other types of information structured for supporting the various activities and information structures described herein. The memory 412 may be coupled to the processor 414 and include computer code or instructions for executing one or more processes described herein.

The processor 414 may be implemented as one or more server processors, application specific integrated circuits (ASIC), field programmable gate arrays (FPGAs), digital signal processor (DSP), microprocessors, or other suitable electronic processing components. The server(s) or server computer may be geographically dispersed relative to other server(s) of the identity verification computing system 400. Further, there may be a variety of different types of server(s) included in the identity verification computing system 400 (e.g., application server, database server, catalog sever, virtual private network (VPN) server, communications server, web server, and so on). The memory device may be included with the server(s). The identity verification computing system 400 is configured to run a variety of application programs and store associated data in a database of the memory 412.

The input/output circuit 420 is configured to exchange data, communications, instructions, etc. with an input/output component of the identity verification computing system 400. In some embodiments, the input/output circuit 420 is incorporated into an input/output device. For example, a laptop, desktop, or tablet computer may include the input/output circuit 420 such that the laptop, desktop, or tablet computer is communicably coupled to the identity verification computing system 400. The input/output circuit 420 is configured to receive communications from, and provide communications to, various employees, agents, or operators associated with the identity verification computing system 400.

The network interface circuit 430 is configured to establish communicable connections with other computing systems (e.g., provider computing system 200, the medical record provider computing systems 300, the platform computing systems 500, the user devices 600, and other computing systems), by way of the network 110. The network interface circuit 430 may include program logic that facilitates connection of the identity verification computing systems 400 to the network 110. For example, the network interface circuit 430 may include a combination of a wireless network transceivers (e.g., a NFC transceiver, a Bluetooth transceiver, a Wi-Fi transceiver, etc.) and/or a wired network transceiver (e.g., an Ethernet transceiver). In some arrangements, the network interface circuit 430 includes the hardware and machine-readable media sufficient to support communication over multiple channels of data communication. Further, in some arrangements, the network interface circuit 430 includes cryptography capabilities to establish a secure or relatively secure communication session in which data communicated over the session is encrypted.

The token generator circuit 440 is configured to generate and/or otherwise create access tokens to be used in a token-based authentication to allow the provider computing system 200 to access an application programming interface (API) of the identity verification computing system 400. Furthermore, the token generator circuit 440 is configured to generate access tokens which associatively map to both an expiration (e.g., a lifespan for the access token contained in the token repository 450) and a set of permissions stored in the permissions repository 460.

The token repository 450 is configured to retrievably hold (e.g., in cache memory), store (e.g., in non-transitory memory), categorize, and/or otherwise serve as a repository for information pertaining to access tokens, configuration options associated with the access tokens (e.g., an expiration), systems authorized to access information of the identity verification computing system 400 (e.g., the provider computing system 200), and API tokens provisioned to systems authorized to access information of the identity verification computing system 400 (e.g., the provider computing system 200). Accordingly, the token repository 450 is configured to retrievably store and access information pertaining to access rights of the provider computing system 200 (e.g., defining the specific identity data the provider computing system 200 is authorized to receive for a particular user).

The permissions repository 360 is configured to retrievably hold (e.g., in cache memory), store (e.g., in non-transitory memory), categorize, and/or otherwise serve as a repository for information pertaining to access control rules governing what systems (e.g., the provider computing system 200) can interact with specific API resources of the identity verification computing system 400, and what actions the systems are permitted to perform (e.g., what data the provider computing system 200 is authorized to receive from the identity verification computing system 400).

The identity record repository 470 is configured to retrievably hold (e.g., in cache memory), store (e.g., in non-transitory memory), categorize, and/or otherwise serve as a repository for identity data of users. For example, the identity record repository 470 can include a variety of personal data aimed at securely verifying an individual's identity, such as biometric data (e.g., fingerprints, facial recognition data, iris scans), personal identification information (e.g., full names, dates of birth, addresses, and government-issued identification numbers such as driver's license or passport numbers), photographs, and scanned documents.

Referring to FIG. 5, a block diagram of a platform computing system 500 of FIG. 1 is shown according to an example embodiment. The platform computing system 500 includes a processing circuit 510, an input/output circuit 520, a network interface circuit 530, and a profile repository 540.

The processing circuit 510 includes memory 512 and processor 514. The processing circuit 510 can be configured to perform all functionalities of the platform computing system 500 as described herein.

The platform computing system 500 can be any type of platform computing system that would benefit from receiving an identity verification or health status check of its users from the provider computing system 200. For example, a non-limiting list of the types of platform computing systems that the platform computing system 500 can be include any type of digital platform, a social media platform, a non-social media platform, a dating platform, a travel platform (e.g., hotel, airline, or activity booking platform), an education platform, a child care platform, an elder care platform, a pet-sitting platform, a life insurance platform, a workplace platform, an employer platform, a telehealth platform, an e-prescribing platform, a health record wallet platform, and so on. It will be appreciated that the platform computing system 500 can include any combination of one or more type of platform.

For example, in an embodiment where the platform computing system 500 is a travel platform, the provider computing system 200 can be used to provide a travel company with an identity verification or health status check of a user using the platform computing system 500 to book travel. In this example, the user can use the provider computing system 200 to provide the platform computing system 500 vaccination or other health records that are required for the user to book a certain activity or that is suggested for travel to a certain country.

In another example, in an embodiment where the platform computing system 500 is an elder care platform, the provider computing system 200 can be used to provide an elder care company with an identity verification or health status check of a user using the platform computing system 500 to provide elder care services through the elder care platform. In this example, the user can use the provider computing system 200 to provide the platform computing system 500 vaccination or other health records that are required for the user to provide services through the elder care platform or to work with patients that request vaccinated caregivers or are more at risk of certain types of illness.

In another example, in an embodiment where the platform computing system 500 is an education or camp platform, the provider computing system 200 can be used to provide a school or camp with an identity verification or health status check of a user attending or planning to attend the school or camp or participating in athletics therein. In this example, the user can use the provider computing system 200 to provide the platform computing system 500 vaccination records that are required for the user (or the user's child) to attend the school or camp, or allergy, disability, or other statuses pertaining to a user or user's child that the user wishes to share with the school or camp.

In another example, in an embodiment where the platform computing system 500 is a telehealth or e-prescribing platform, the provider computing system 200 can be used to provide a telehealth or e-prescribing company with an identity verification or health status check of a user using the platform computing system 500 to seek medical care or a prescription. In this example, the user can use the provider computing system 200 to provide the platform computing system 500 prescription medication records or other health status data in lieu of completing medical history questionnaires or requesting other medical service providers to provide health records to the telehealth or e-prescribing company, which would otherwise result in delays or the unsecured sharing of confidential health information.

It will be appreciated that these examples can apply to any type of digital platform making use of the systems and methods disclosed herein for unique use cases requiring or benefiting from verified identities or the receipt of health status data particular to that industry.

The memory 512 may be one or more devices (e.g., RAM, ROM, Flash memory, hard disk storage) for storing data and/or computer code for completing and/or facilitating the various processes described herein. Memory 512 may be or include non-transient volatile memory, non-volatile memory, and non-transitory computer storage media. Memory 512 may include database components, object code components, script components, or other types of information structured for supporting the various activities and information structures described herein. The memory 512 may be coupled to the processor 514 and include computer code or instructions for executing one or more processes described herein.

The processor 514 may be implemented as one or more server processors, application specific integrated circuits (ASIC), field programmable gate arrays (FPGAs), digital signal processor (DSP), microprocessors, or other suitable electronic processing components. The server(s) or server computer may be geographically dispersed relative to other server(s) of the platform computing system 500. Further, there may be a variety of different types of server(s) included in the platform computing system 500 (e.g., application server, database server, catalog sever, virtual private network (VPN) server, communications server, web server, and so on). The memory device may be included with the server(s). The platform computing system 500 is configured to run a variety of application programs and store associated data in a database of the memory 512.

The input/output circuit 520 is configured to exchange data, communications, instructions, etc. with an input/output component of the platform computing system 500. In some embodiments, the input/output circuit 520 is incorporated into an input/output device. For example, a laptop, desktop, or tablet computer may include the input/output circuit 520 such that the laptop, desktop, or tablet computer is communicably coupled to the platform computing system 500. The input/output circuit 520 is configured to receive communications from, and provide communications to, various employees, agents, or operators associated with the platform computing system 500.

The network interface circuit 530 is configured to establish communicable connections with other computing systems (e.g., provider computing system 200, the medical record provider computing systems 300, the identity verification computing systems 400, the user devices 600, and other computing systems), by way of the network 110. The network interface circuit 530 may include program logic that facilitates connection of the platform computing systems 500 to the network 110. For example, the network interface circuit 530 may include a combination of a wireless network transceivers (e.g., a NFC transceiver, a Bluetooth transceiver, a Wi-Fi transceiver, etc.) and/or a wired network transceiver (e.g., an Ethernet transceiver). In some arrangements, the network interface circuit 530 includes the hardware and machine-readable media sufficient to support communication over multiple channels of data communication. Further, in some arrangements, the network interface circuit 530 includes cryptography capabilities to establish a secure or relatively secure communication session in which data communicated over the session is encrypted.

The profile repository 540 is configured to retrievably hold (e.g., in cache memory), store (e.g., in non-transitory memory), categorize, and/or otherwise serve as a repository for identity data of users. For example, the profile repository 540 can include a wide array of data on its users, encompassing both personal information and activity-related data, including user profile data (e.g., names, email addresses, phone numbers, birthdates, profile pictures), demographic details (e.g., gender, location, interests), social connections (e.g., friends, followers, group memberships), communication records (e.g., messages, comments, interactions), posts, photos, videos, check-ins, shared content, engagement metrics (e.g., likes, reactions, and shares), behavioral data (e.g., browsing patterns, ad clicks, interaction history), device information, location data, and usage logs.

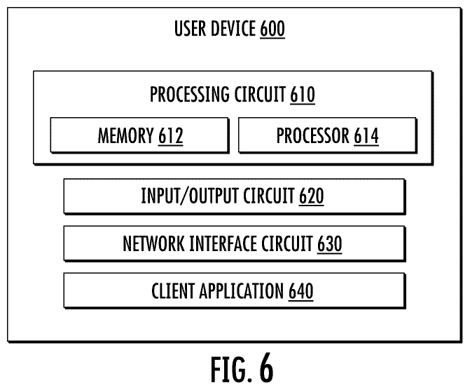

Referring to FIG. 6, a block diagram of a user device 600 of FIG. 1 is shown according to an example embodiment. The user device 600 includes a processing circuit 610, an input/output circuit 620, a network interface circuit 630, and a client application 640.

The processing circuit 610 includes memory 612 and processor 614. The processing circuit 610 can be configured to perform all functionalities of the user device 600 as described herein.

The memory 612 may be one or more devices (e.g., RAM, ROM, Flash memory, hard disk storage) for storing data and/or computer code for completing and/or facilitating the various processes described herein. Memory 612 may be or include non-transient volatile memory, non-volatile memory, and non-transitory computer storage media. Memory 612 may include database components, object code components, script components, or other types of information structured for supporting the various activities and information structures described herein. The memory 612 may be coupled to the processor 614 and include computer code or instructions for executing one or more processes described herein.

The processor 614 may be implemented as one or more server processors, application specific integrated circuits (ASIC), field programmable gate arrays (FPGAs), digital signal processor (DSP), microprocessors, or other suitable electronic processing components. The server(s) or server computer may be geographically dispersed relative to other server(s) of the user device 600. Further, there may be a variety of different types of server(s) included in the user device 600 (e.g., application server, database server, catalog sever, virtual private network (VPN) server, communications server, web server, and so on). The memory device may be included with the server(s). The user device 600 is configured to run a variety of application programs and store associated data in a database of the memory 612.

The input/output circuit 620 is configured to receive communications from and provide communications to a user via the user device 600. In this regard, the input/output circuit 620 is configured to exchange data, communications, instructions, etc. with an input/output component of the user device 600. In some embodiments, the input/output circuit 620 includes an input/output device. In some embodiments, the input/output circuit 620 includes communication circuitry for facilitating the exchange of data, values, messages, and the like between an input/output device and the components of the user device 600. In some embodiments, the input/output circuit 620 includes machine-readable media for facilitating the exchange of information between an input/output device and the components of the user device 600. In some embodiments, the input/output circuit 620 includes a combination of hardware components, communication circuitry, and machine-readable media.

For example, in some embodiments, the input/output circuit 620 may include suitable input/output ports and/or use an interconnect bus (not shown) for interconnection with a local display (e.g., a touchscreen display) and/or keyboard/mouse devices (when applicable), or the like, serving as a local user interface for programming and/or data entry, retrieval, or manipulation purposes. That is, the input/output circuit 620 provides an interface for a user to interact with various applications (e.g., the client application 640) accessed by the user device 600.

The network interface circuit 630 is configured to establish communicable connections with other computing systems (e.g., provider computing system 200, the medical record provider computing systems 300, the identity verification computing systems 400, the platform computing systems 500, and other computing systems), by way of the network 110. The network interface circuit 630 may include program logic that facilitates connection of the user device 600 to the network 110. For example, the network interface circuit 630 may include a combination of a wireless network transceivers (e.g., a NFC transceiver, a Bluetooth transceiver, a Wi-Fi transceiver, etc.) and/or a wired network transceiver (e.g., an Ethernet transceiver). In some arrangements, the network interface circuit 630 includes the hardware and machine-readable media sufficient to support communication over multiple channels of data communication. Further, in some arrangements, the network interface circuit 630 includes cryptography capabilities to establish a secure or relatively secure communication session in which data communicated over the session is encrypted.

The client application 640 is provided by and coupled to the provider computing system 200. In some embodiments, the client application 640 may be a standalone application or be incorporated with an existing application of the user device 600 (e.g., integrated into a social media platform application or other digital platform, etc.). The client application 640 may be downloaded by the user device 600 prior to its usage, hard coded into the memory 612 of the user device 600, or be a network-based or web-based interface application such that the provider computing system 200 can provide a web browser to access the application, which may be executed remotely from the user device 600. In the example shown, the client application 640 is downloaded to the user device 600 and provided by the provider computing system 200 via, for example, an app store for download. In the example shown, the client application 640 is configured as an provider application, though it will be appreciated that the client application 640 can be configured as just a standalone identity check application, a standalone health check application, or any combination of an identity check application, a health check application, and other types of applications. The client application 640 may be developed and maintained (e.g., provided with software updates on a regular or semi-regular basis) by the provider computing system 200 using the provider computing system 200. Accordingly, the user device 600 may include software and/or hardware capable of implementing a network-based or web-based application. For example, in some instances, the client application 640 includes software such as HTML, XML, WML, SGML, PHP (Hypertext Preprocessor), CGI, and like languages.

In the latter web-based instance, the user may have to log onto or access the web-based interface before usage of the application. Further, and in this regard, the client application 640 may be supported by the provider computing system 200 via one or more servers, processors, network interface circuits, etc. that transmit applications for use to the user device 600. Furthermore, prior to use of the client application 640 and/or at various points throughout the use of the client application 640, the user may be required to provide various authentication information or log-in credentials (e.g., a password, a personal identification number (PIN), a fingerprint scan, a retinal scan, a voice sample, a face scan, any other type of biometric security scan) to ensure that the user associated with the user device 600 is authorized to use the client application 640.

The client application 640 is configured to provide displays (e.g., generated by the input/output circuit 620 of the user device 600 or generated by the provider computing system 200 and transmitted over the network 110) to the user device 600 to provide information pertaining to provider statuses (e.g., as described further herein).

It will be appreciated that the user device 600 can be operated, owned, possessed, or otherwise under the control of a user of the system 100, and the user can be a person, company, organization, government entity, or other entity or individual.

Referring to FIG. 7, a flow chart illustrating a method 700 for providing a health status verification is shown according to an example embodiment. In some embodiments, the provider computing system referred to by method 700 is the provider computing system 200 described above with reference to FIG. 2, such that method 700 may be implemented by the provider computing system 200. In some embodiments, method 700 may be implemented as executable instructions in a memory of the provider computing system 200, such as the memory 212 of FIG. 2.

Step 705 of method 700 can include receiving personal data of a user. In some embodiments, the personal data of the user is received via a user device (e.g., user device 600) associated with the user. The personal data can include any data necessary for the creation of an account for the user with the provider computing system 200, including demographic data. For example, the personal data can include the user's first name, the user's last name, the user's date of birth, a photograph of the user (e.g., a selfie photograph, a photograph of a driver's license), a location of the user (e.g., residence city and state), and a desired username for the account, among other information.

Step 710 of method 700 can include creating a user account associated with the provider computing system 200 for the user based on the personal data of the user. Upon creation of the account for the user, the user may be able to configure their account. For example, the user may be able to provide a number of preferences for their account. In another example, the user is able to identify any number of medical record provider computing systems 300 that the user has medical records through, and authorize the provider computing system 200 to obtain medical data from.

In some embodiments, the user may be able to select preferences regarding specific health data they would like to have verified and communicated to the platform computing system 500. For example, in some embodiments, the user can select preferences to authorize the provider computing system 200 to verify, and provide to a third party such as the platform computing system 500, their HIV status or other test results, family medical history, a vaccination status (e.g., a COVID vaccination status), whether the user has been prescribed certain medications among other health conditions or health data. As such, the provider computing system 200 enables the user to be in control of what specific health data is accessible by the provider computing system 200, and what specific health data the provider computing system 200 is authorized to publish or to share with third parties, such as the platform computing system 500.

In some embodiments, the user is able to select how often the provider computing system 200 is authorized to request their health data from the medical record provider computing system 300. For example, the user can authorize the provider computing system 200 to request their health data in real time, based on an interval (e.g., every month, every other month, every three months, etc.), whenever the user logs into the client application 640 associated with the provider computing system 200, or a single instance. Accordingly, the provider computing system 200 enables the user to be in control of when and how their health data is accessed and shared. The user can provide such authorization using at least one of the provider computing system 200, the medical record provider computing system 300, or the user device 600. For example, the user can make a selection regarding an authorization to provide their health data to the provider computing system 300 via the medical record provider computing system 300 (e.g., by logging into the medical record provider computing system 300 using the user device 600 or another device, or calling an entity associated with the medical record provider computing system 300 and providing their authorization to share data and specify a frequency for the medical record provider computing system 300 to share data with the provider computing system 200).

Step 715 of method 700 can include receiving identity verification information of the user from the identity verification computing system 400. In some embodiments, the identity verification information of the user is received via a first application programming interface (API). The identity verification information of the user can include a biometric of the user (e.g., at least one of a name of the user, an age of the user, a photograph of the user, etc.). It will be appreciated that the identity verification computing system 400 can be one or more of any available identity verification computing systems, in some embodiments, provided by a third-party identity verification service provider.

In some embodiments, the identity verification computing system 400 performs its own process to verify the user's identity. For example, the identity verification computing system 400 can require the user to upload a photograph of a government-issue identification card, such as a driver's license. In another example, the identity verification computing system 400 can require the user to upload one or more photographs of themselves, such as selfie photographs. In another example, the identity verification computing system 400 can require the user to provide additional identifying information, such as a home address, a social security number, or other information. In some examples, the identity verification computing system 400 can require the user to provide any combination of the above items.

While the identity verification computing system 400 may have access to additional identifying information of the user, the identity verification computing system 400 can provide limited identifying information of the user back to the provider computing system 200. For example, the identity verification computing system 400 may provide the provider computing system 200 with a verification of the user's name, the user's date of birth, the user's home address, and an image of the user.

In some embodiments, step 715 is skipped. For example, if the provider computing system 200 verifies the user's identity another way, the step of receiving additional identity verification information from an identity verification computing system 400 is not performed. In some embodiments, the user can upload a new photograph of themselves (e.g., a selfie or other photograph showing their face) directly to the provider computing system 200 via the client application 640. In this example, when the user uploads a new photograph, the provider computing system 200 is configured to verify that the new photograph is indeed of the user by comparing a face in the new photograph to an image received from the identity verification computing system 400 (e.g., an image of the a government-issued identification card, a selfie photograph or other photograph of the user submitted to the identity verification computing system 400 during the identity verification process conducted at step 715). Upon the provider computing system 200 verifying that the new photograph indeed does include a picture of the user, the provider computing system 200 enables the new photograph to be displayed via the client application 640 or via a profile associated with the platform computing system 500. In some embodiments, the provider computing system 200 enables an identity verification badge or other indicia to be displayed with the new photograph of the user.

In some embodiments, the provider computing system 200 is configured to verify the identity of the user without receiving verification from the identity verification computing system 400.

Step 720 of method 700 can include providing a request for health data of the user to a medical record provider computing system 300. In some embodiments, the request for health data of the user is provided to the medical record provider computing system 300 via a second API. The provider computing system 200 can request the health data based on the selected preferences of the user. For example, provider computing system 200 can request the health data based on a frequency selected by the user (e.g., in real time, based on an interval, whenever the user logs into the client application 640, a single instance). In some embodiments, the second API is the Fast Healthcare Interoperability Resources (FHIR) API, though it will be appreciated that any API can be used.

For example, for the provider computing system 200 to obtain data from the medical record provider computing system 300 using an API, the provider computing system 200 follow a series of steps, beginning with identifying the API endpoint and its requirements. The provider computing system 200 first determines which API endpoint on the medical record provider computing system 300 it needs to access, what kind of authentication is required, and which HTTP methods (e.g., GET or POST) and parameters should be used. The provider computing system 200 then obtains credentials for authentication, such as an API key received from the medical record provider computing system 300 or OAuth 2.0 credentials, such as a client ID and client key.

After obtaining the credentials, the provider computing system 200 authenticates the request. For example, if the API requires an API key, the provider computing system 200 can include the API key in the headers or query string of each request. In another example, if the API uses OAuth 2.0, the provider computing system 200 first authenticates with an authorization server of the medical record provider computing system 300. The provider computing system 200 can send a request containing the client ID, client key, and scope (e.g., defining the data requested by the provider computing system 200). If the credentials are correct, the authorization server provides an access token, which the provider computing system 200 can use in future API calls. In some embodiments, the access token is provided with a refresh token that enables the provider computing system 200 to renew the access token when it expires.

To provide a request to the medical record provider computing system 300, the provider computing system 200 sends the request, including the API access token, to the appropriate API endpoint (in cases where an API key is used instead of a token, the key can be sent as a query parameter). Upon receiving the request, the medical record provider computing system 300 validates the access token to ensure the token is legitimate, hasn't expired, and grants the provider computing system 200 the proper permissions to access the requested resource.

After validating the token, the medical record provider computing system 300 processes the request by retrieving data from a database (e.g., medical record repository 370), performing calculations, or gathering information from another source. Once the medical record provider computing system 300 fulfills the request, the medical record provider computing system 300 returns the requested data (e.g., the health data) to the provider computing system 200.

In some embodiments, if the token has expired or is invalid, the medical record provider computing system 300 returns an unauthorized response message to the provider computing system 200. In this example, the provider computing system 200 can then re-authenticate to obtain a new access token, or request a new access token without requiring re-authentication using a refresh token. In some embodiments, if the refresh token has expired, the user must log into the client application 640 to reauthenticate.

Step 725 of method 700 can include receiving the health data of the user from the medical record provider computing system 300. In some embodiments, the health data is received from the medical record provider computing system 300 via the second API. In some embodiments, the health data is received in the form of medical codes and related values, such as codes in using the Logical Observation Identifiers Names and Codes (LOINC) format, the Systematized Nomenclature of Medicine Clinical Terms (SNOMED CT) codes, or RxNorm, though it will be appreciated that the health data can be received in any format. The health data received by the provider computing system 200 is limited to the data requested in the API request and can be defined by the user preferences dictating what specific health data the provider computing system 200 is authorized by the user to receive. As such, the health data is limited to certain categories of health data or certain medical codes. In some embodiments, the health data includes lab results, diagnosis, and treatment data. In some embodiments, the health data received by the provider computing system 200 includes sexual wellness data, HIV status, family medical history, a vaccination status (e.g., a COVID vaccination status), whether the user is taking certain medication (e.g., pre-exposure prophylaxis medication), among other health conditions or other health data. It will be appreciated that the health data can include any kind of health data relating to any possible diagnosis, rest result, opinion, etc. Upon receiving the health data, the provider computing system 200 stores the health data in a memory, such as the user data repository 240, or in a third-party server, such as a Fast Healthcare Interoperability Resources (FHIR) server controlled by the provider computing system 200. In some embodiments, the provider computing system 200 uses the identity verification information obtained via the first API to confirm ownership of the health data obtained via the second API. For example, the provider computing system 200 can match the received identity verification information with corresponding data originating from the medical record provider community system 300 (for example, name, date of birth, and/or home address, to confirm that the identity of the individual from the identity verification information matches the identity of the individual from the health data (e.g., to ensure the health data is indeed heath data of the same individual whose identity has been verified).

Step 730 of method 700 can include determining a health status of the user based on the health data. The provider computing system 200 determines the health status of the user by interpreting the received medical data (e.g., medical codes, lab results, diagnoses). In an embodiment, the provider computing system 200 uses a logic-driven translation layer to determine a health status of the user. Put another way, the provider computing system 200 can transform clinical health data, such as medical codes, into human-readable health statuses for secure sharing via digital platforms. For example, the provider computing system 200 can interpret LOINC SNOMED CT, and RxNorm medical codes (and any related values) received with the health data to determine a health status of the user. For example, the provider computing system 200 can interpret the health data to determine an HIV status (e.g., negative, positive, positive undetectable), dates of most recent HIV or sexually transmitted infection (STI) screenings, status of STI infections and treatments, active prescriptions for medication to prevent HIV and other infections or diseases, vaccination history (e.g., COVID vaccinations, other vaccinations), and other data relevant to sexual or general health.

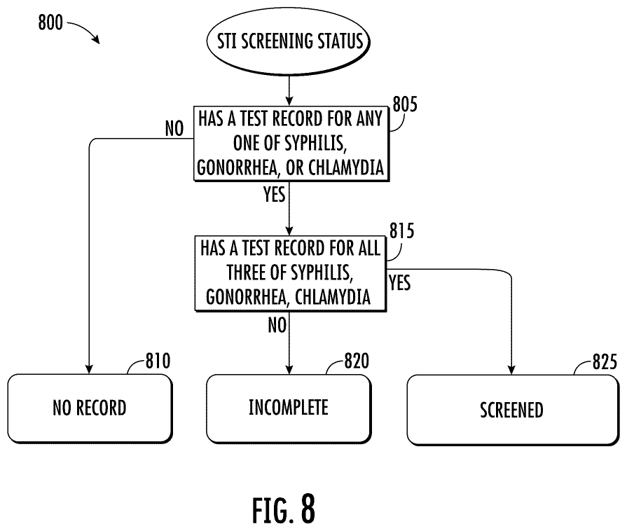

Examples of processes the provider computing system 200 can use to determine health statuses are illustrated and described with reference to FIGS. 8-11.

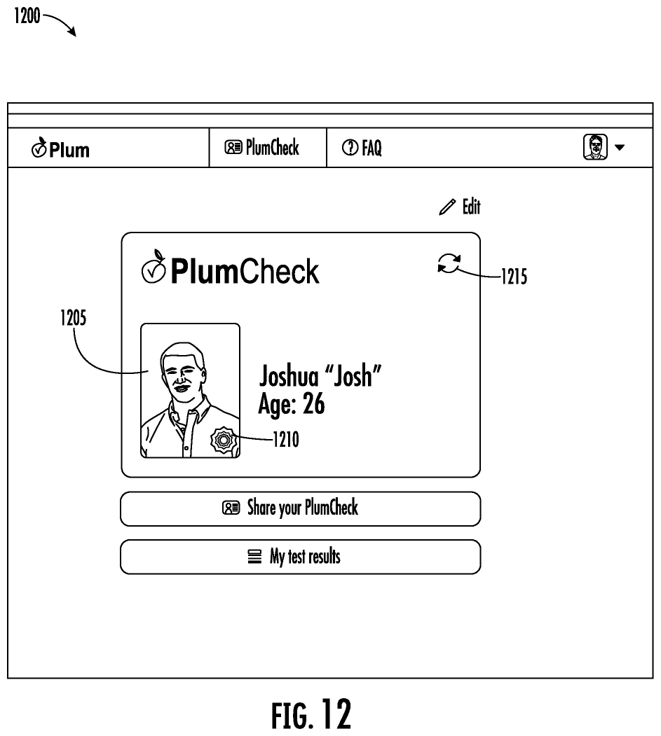

Step 735 of method 700 can include converting the health status of the user into one or more display codes. The provider computing system 200 determines a display code associated with the determined health status of the user. The display code is stored in a memory of the provider computing system 200 (e.g., memory user data repository 240). The display code is associated with a graphical or text user interface element indicating the health status. In some embodiments, each health status determined by the provider computing system 200 is associated with a unique and dedicated display code. For example, a unique display code can exist for each badge 1210, 1410, 1510, 1610 or user interface element 1325, 1330, 1335, 1340, 1345, 1350, 1515, 1520, 1725, 1730, 1735, 1740, 1745, 1750 shown in FIGS. 12-17.

Each display code can be associated with a graphical user interface element or icon, such as a “badge” that is displayable in the client application 640 or in a user profile of the user managed or hosted by the platform computing system 500. For example, an HIV negative code can be displayed as a badge with a ribbon and “−” mark or a related symbol as directed by or approved by client of the provider computing system 200 (e.g., any number of platforms associated with a platform computing system, such as the platform computing system 500). In another example, specific codes and values can be associated with other corresponding STIs (e.g., gonorrhea, syphilis, chlamydia, etc.), medication confirmations (e.g., a status of taking or being prescribed a pre-exposure prophylaxis (PrEP), blood pressure medication, etc.), or vaccination status (e.g., status of having had a vaccine for COVID, etc.). In some embodiments, a display code can be based on multiple health statuses. For example, a sexual wellness check badge can be based on the user having negative test results, or being adequately treated (e.g., according to a medical guideline such as a U.S. Center of Disease Control (CDC) guideline), for each of gonorrhea, syphilis, and chlamydia, or other medical condition.

In this way, the provider computing system 200 is able to determine standardized health statuses based, for example, on recent STI screenings, treatments, and vaccinations for a plurality of users of the client application 640 or one or more platforms associated with platform computing systems 500, which provides consistent, reliable, trusted, and easily understood summaries of a user's recent sexual health or overall health. The system also makes medical information accessible to non-medical users, lowering the threshold of communication about sexual health and other health topics.

Step 740 of method 700 can include causing the health status of the user to be displayed on at least one of a user device 600 of the user via a first instance of the client application 640, a third-party user device 600 of a third party via a second instance of the client application 640, or via the platform computing system 500. In some embodiments, the provider computing system 200 is configured to provide the health status to a plurality of third-party computing systems, such as platform computing system 500 so that the health status can be displayed by each third-party computing system.

In some embodiments, the health status is displayed in the form of text. For example, the system can display text or indicia on user interface profiles displayed via the client application 640 or a user profile via the platform computing systems 500 as shown in FIGS. 12-17.

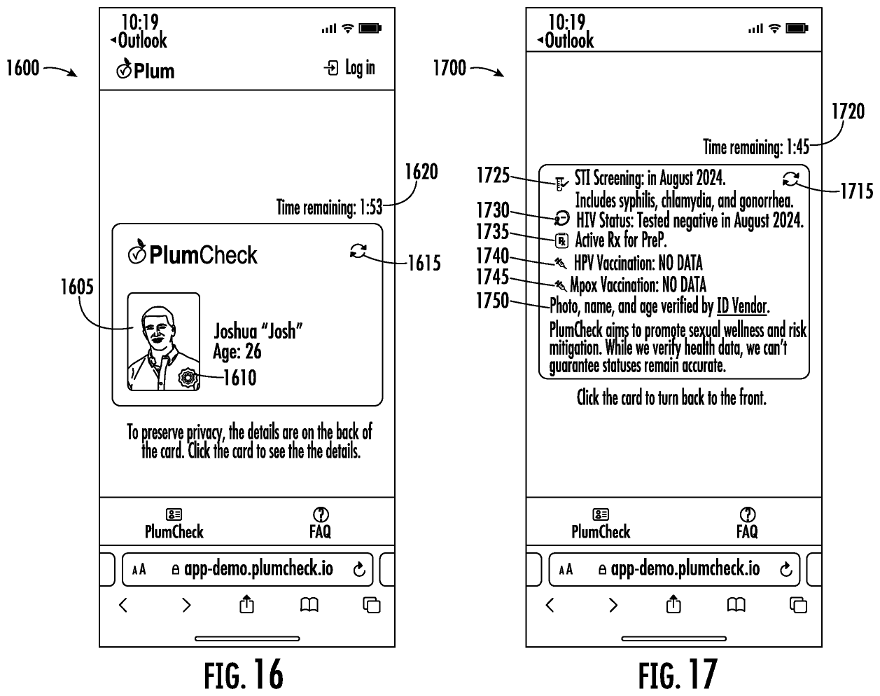

In some embodiments, the user can provide access to their profile displaying their sexual health statuses (e.g., via the client application 640) by sending a message including a link to another user. In some embodiments, the message can be sent via text message, email, or chat. In some embodiments, the link is configured to direct the other user to a third-party website or application or to a client website or the client application 640, which then displays the user's profile including their health statuses. In some embodiments, the link expires after a set time period (e.g., 1 hour, 2 hours, 12 hours, 24 hours), and once the other user accesses the user's profile via the link, the other user can only view the user's profile for a set time period (e.g., 1 minute, 2 minutes, 3 minutes, 5 minutes, 1 hour, 2 hours, etc.). In some embodiments, the link does not expire or the other user can view the user's profile without time period limitations (e.g., as shown in FIGS. 16-17).

Step 745 of method 700 can include destroying the health status data. The provider computing system 200 destroys the health data received from the medical record provider computing systems 300 after determining the display codes for the user, thereby reducing the risk of user health data being exposed to the general public, ensuring confidentiality of the health data, and providing users assurances that their health data will not be used for other purposes or commercialized. In some embodiments, the provider computing system 200 can destroy the health data and health status data of the user after a predetermined time period. For example, the provider computing system 200 can destroy the health data and health status data of the user anytime within the range of 1 day to 1 year. For example, the provider computing system 200 can destroy the health data and health status data of the user after 30 days, after 60 days, after 90 days, etc.

Step 750 of method 700 can include obtaining updated health data of the user to determine an updated health status of the user and one or more updated display codes. The provider computing system 200 can renew the health data (e.g., similar or the same as steps 720 and 724). In some embodiments, the provider computing system 200 renews the health data by sending new requests over the second API to one or more of the medical record provider computing systems 300. The provider computing system 200 can renew a user's health data based on a user preference set by the user, or based on a default setting. For example, the provider computing system 200 can renew a user's health data continuously and in real time, based on an interval (e.g., every month, every other month, every three months, etc.), whenever the user logs into the client application 640 associated with the provider computing system 200, or a single instance. Accordingly, the provider computing system 200 enables the user to be in control of when and how their health data is accessed and shared.

After renewing the user's health data, the provider computing system 200 is configured to determine an updated health status of the user based on the updated health data (e.g., similar to or the same as step 730), determine an updated display code associated with the updated health status of the user (e.g., similar to or the same as step 735), and replace the display code with the updated display code in the memory and causing the updated display code to be displayed in association with the user's profile via the client application 640 or via the platform computing systems 500 (e.g., similar to or the same as step 740). Accordingly, the provider computing system 200 can store the most up-to-date display data and destroy any historical display data superseded by newer data. In some embodiments, the provider computing system 200 can store all or select health data received from the medical record provider computing systems 300, and all or select health statuses determined by the provider computing system 200.

Referring to FIG. 8, a flow chart illustrating a method 800 for determining a sexually transmitted infection (STI) health status of a user is shown according to an example embodiment. In some embodiments, method 800 may be implemented by the provider computing system 200. In some embodiments, method 800 may be implemented as executable instructions in a memory of the provider computing system 200, such as the memory 212 of FIG. 2.

Step 805 of method 800 can include determining if the health data from the medical record provider computing system 300 is indicative of the user having a test record for any STI. For example, step 805 can include determining if the health data from the medical record provider computing system 300 is indicative of the user having a test record for any one or more of syphilis, gonorrhea, or chlamydia, though it will be appreciated that the determination can take into consideration test records for any number of sexually transmitted infections. If at step 805 no record is identified, the method 800 advances to step 810, which may cause the various user interfaces displaying health status check data of the user to indicate the user has no test records for such STIs. If at step 805 a record is identified, then the method 800 advances to step 815.

Step 815 of method 800 can include determining if the health data from the medical record provider computing system 300 is indicative of the user having a test record for a predetermined set of STIs (e.g., syphilis, gonorrhea, and chlamydia). If at step 815 the user is determined to not have a test record for each of the STIs of the predetermined set of STIs, then the method 800 advances to step 820 indicating the user's STI test results are incomplete, which may cause the various user interfaces displaying health status check data of the user to indicate the user has incomplete STI test records. If at step 815 the user is determined to have a rest record for each of the STIs of the predetermined set of STIs, then the method 800 advances to step 820 indicating the user is screened, which may cause the various user interfaces displaying health status check data of the user to indicate the user has complete STI test records and to display the last date the user was tested for each STI, or to display the earliest of the test administration dates of the set of STI test records. For example, if the user was tested for syphilis on Jun. 25, 2024, was tested for gonorrhea on May 25, 2024, and tested for Chlamydia on Jun. 25, 2025, the date associated with the user's STI health check data would be the earliest of those dates (i.e., May 25, 2024).

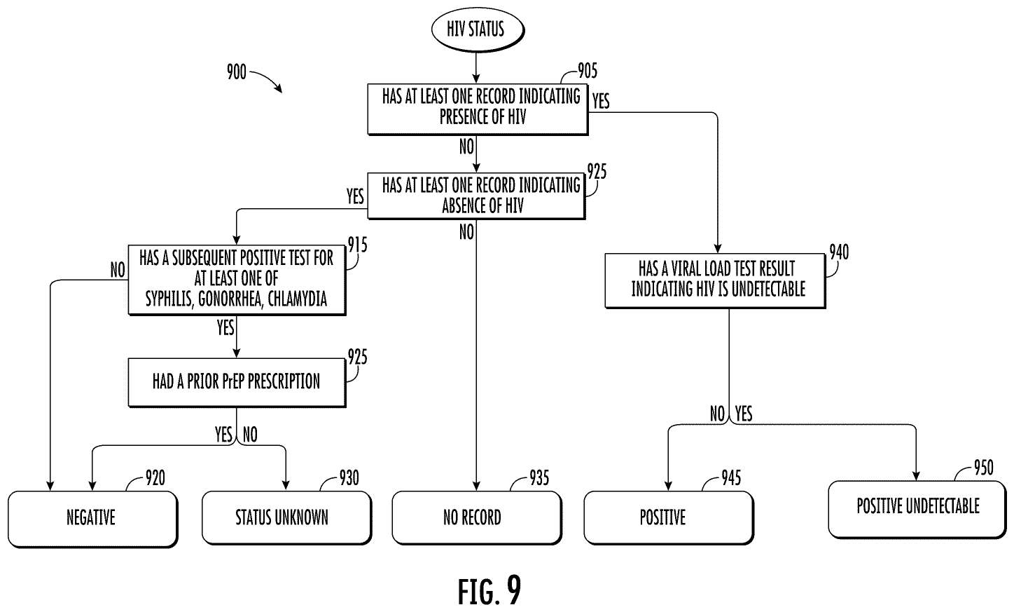

Referring to FIG. 9, a flow chart illustrating a method 900 for determining a human immunodeficiency virus (HIV) health status of a user is shown according to an example embodiment. In some embodiments, method 900 may be implemented by the provider computing system 200. In some embodiments, method 900 may be implemented as executable instructions in a memory of the provider computing system 200, such as the memory 212 of FIG. 2.

Step 905 of method 900 can include determining if the health data from the medical record provider computing system 300 is indicative of the user having a test record indicating a presence of HIV. For example, the determination can include determining if the user's health data is indicative of an FHIR condition record with relevant ICD-10 code, a positive Ag/Ab test, or a viral load test with a specific value above 0. If at step 905 no relevant test record is identified indicative of the user having a test record indicating a presence of HIV, the method 900 advances to step 910. If at step 905 a relevant test record is identified indicative of the user having a test record indicating a presence of HIV, the method 900 advances to step 940.

Step 910 of method 900 can include determining if the health data from the medical record provider computing system 300 is indicative of the user having a test record indicating an absence of HIV. For example, the determination can include determining if the user's health data is indicative of a negative Ag/Ab test or a viral load test with a value of 0. If at step 910 a relevant test record is identified indicative of the user having a test record indicating an absence of HIV, the method 900 advances to step 915. If at step 910 a relevant test record is identified indicative of the user having no test record indicating an absence of HIV, the method 900 advances to step 935, which is indicative of the user having no record of a test record indicating an absence of HIV and which may cause the various user interfaces displaying health status check data of the user to indicate the user has no record of an absence of HIV.

Step 915 of method 900 can include determining if the health data from the medical record provider computing system 300 is indicative of the user having a subsequent positive test for an STI. For example, step 915 can include determining if the health data from the medical record provider computing system 300 is indicative of the user having a test record for any one or more of syphilis, gonorrhea, or chlamydia, though it will be appreciated that the determination can take into consideration test records for any number of sexually transmitted infections. If at step 915 no relevant test record is identified, the method 900 advances to step 920, which is indicative of the user being negative for HIV and which may cause the various user interfaces displaying health status check data of the user to indicate the user is HIV negative and to display the most recent date that the user tested negative for HIV and to display a pre-exposure prophylaxis (PrEP) status of the user. If at step 915 a relevant test record is identified, the method 900 advances to step 925.

Step 925 of method 900 can include determining if the health data from the medical record provider computing system 300 is indicative of the user having a prior PrEP prescription. If at step 925 it is determined that the user had a prior PrEP prescription, the method 900 advances to step 920, which is described above. If at step 925 it is determined that the user had no prior PrEP prescription, the method 900 advances to step 930, which is indicative of determining an unknown HIV status of the user and which may cause the various user interfaces displaying health status check data of the user to indicate the user has an unknown HIV status.

Step 940 of method 900 can include determining if the health data from the medical record provider computing system 300 is indicative of the user having a viral load test result indicating HIV is undetectable. For example, the determination can include determining if the user's health data is indicative of a viral test load having a specific value below a threshold (e.g., 200 copies/mL) or a range below a threshold (e.g., a range below 200 copies/mL, such as less than 200 or less than 50). If at step 940 it is determined that the user does not have a viral load test result indicating HIV is undetectable, the method 900 advances to step 945, which is indicative of the user being HIV positive and which may cause the various user interfaces displaying health status check data of the user to indicate the user is HIV positive. If at step 940 it is determined that the user does have a viral load test result indicating HIV is undetectable, the method 900 advances to step 950, which is indicative of the user having an undetectable HIV positive test result and which may cause the various user interfaces displaying health status check data of the user to indicate the user has an undetectable HIV positive test result and to display the date of the user's most recent test indicating that HIV is undetectable.

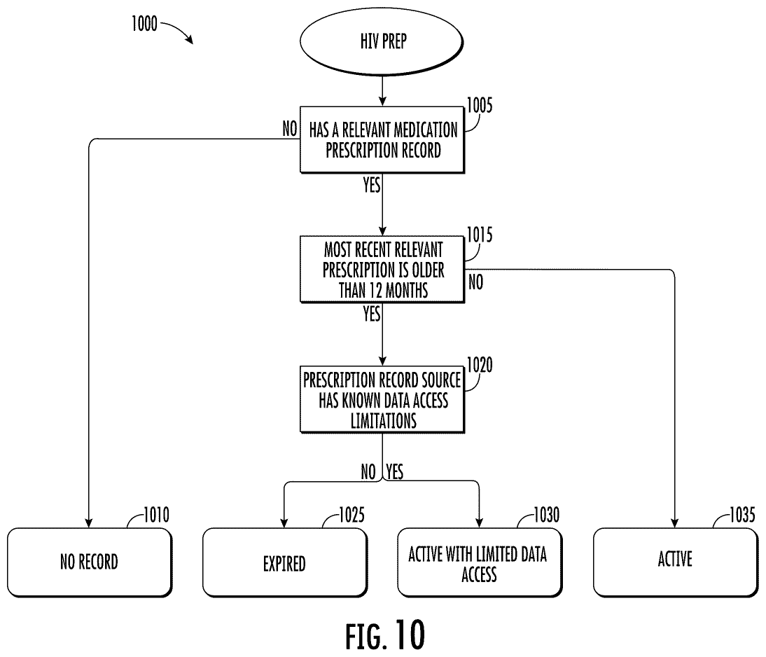

Referring to FIG. 10, a flow chart illustrating a method 1000 for determining a pre-exposure prophylaxis (PrEP) health status of a user is shown according to an example embodiment. In some embodiments, method 1000 may be implemented by the provider computing system 200. In some embodiments, method 1000 may be implemented as executable instructions in a memory of the provider computing system 200, such as the memory 212 of FIG. 2.

Step 1005 of method 1000 can include determining if the health data from the medical record provider computing system 300 is indicative of the user having a relevant medical prescription record for a PrEP. If at step 1005 it is determined the user does not have a relevant medical prescription record for a PrEP, the method 1000 advances to step 1010, which is indicative of the user not having a relevant medical prescription record for a PrEP, which may cause the various user interfaces displaying health status check data of the user to indicate the user does not have a relevant medical prescription record for a PrEP. If at step 1005 it is determined the user does have a relevant medical prescription record for a PrEP, the method 1000 advances to step 1015.

Step 1015 of method 1000 can include determining if the health data from the medical record provider computing system 300 is indicative of the user's most recent prescription for a PrEP is older than 12 months. If at step 1015 it is determined that the user's most recent prescription for a PrEP is older than 12 months, the method 1000 advances to step 1020. If at step 1015 it is determined that the user's most recent prescription for a PrEP is not older than 12 months, the method 1000 advances to step 1035, which is indicative of the user's PrEP prescription status of being active, which may cause the various user interfaces displaying health status check data of the user to indicate the user has an active PrEP prescription and may display the most recent prescription date.

Step 1020 of method 1000 can include determining if the health data from the medical record provider computing system 300 has known data access limitations. If at step 1020 it is determined that the health data from the medical record provider computing system 300 does not have known data access limitations, the method 1000 advances to step 1025, which is indicative of the user's PrEP prescription status as being expired, which may cause the various user interfaces displaying health status check data of the user to indicate the user has an expired PrEP prescription. If at step 1020 it is determined that the health data from the medical record provider computing system 300 does have known data access limitations, the method 1000 advances to step 1030, which is indicative of the user's PrEP prescription status as being active with limited data access, which may cause the various user interfaces displaying health status check data of the user to indicate the user has an active PrEP prescription but that prescription data access is limited and may further display the most recent prescription date for the PrEP prescription.

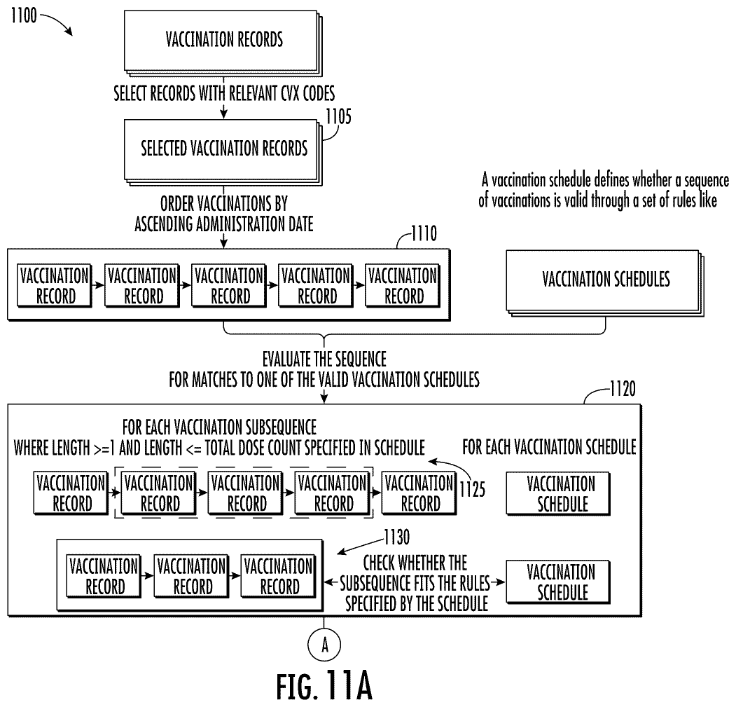

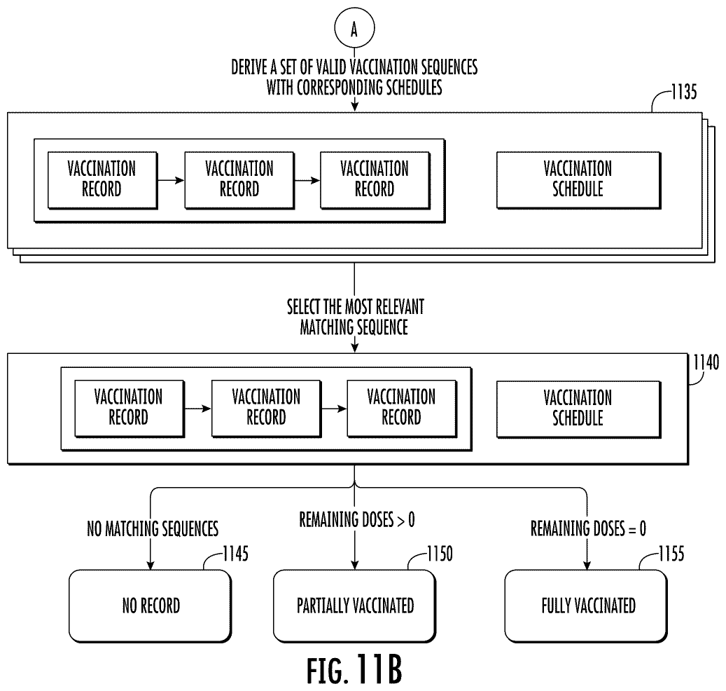

Referring to FIGS. 11A-11B, a flow chart illustrating a method 1100 for determining a vaccination health status of a user is shown according to an example embodiment. In some embodiments, method 1100 may be implemented by the provider computing system 200. In some embodiments, method 1100 may be implemented as executable instructions in a memory of the provider computing system 200, such as the memory 212 of FIG. 2.

Step 1105 of method 1100 can include selecting vaccination records from the health data from the medical record providing computing system 300. The selection may include only relevant vaccination records, meaning only the vaccination records that are associated with health status check data that is ultimately displayed on one of the various user interfaces displaying health status check data of the user.

Step 1110 of method 1100 can include ordering the selected vaccination records. The ordering may be based on an ascending administration date of the vaccination per the vaccination records.

Step 1115 of method 1100 can include determining a vaccination schedule for various vaccinations. In an embodiment, step 1115 includes determining the vaccination schedules for the vaccination records selected during step 1105. In some embodiments, the vaccination schedules define whether a sequence of vaccinations is valid based on a set of rules, for example, such as a total dose count, accepted patient ages, age intervals for each vaccination, accepted time intervals between vaccinations, the absence of certain health conditions, or the presence of certain health conditions. For example, for an HPV vaccination, Gardasil-9, the vaccination schedule may require, for an effective vaccination, three vaccinations in total where the first dose is administered at age 15 or older and the patient has no record of HIV infection.

Step 1120 of method 1100 can include evaluating the ordered selected vaccination records from step 1110 and the vaccination schedules from 1115 to determine whether the selected vaccinations have been properly administered. Step 1120 may include determining, at step 1125 and for each vaccination subsequence where a length is greater than or equal to 1 and less than or equal to a total dose count specified in the vaccination schedule, a subset of the ordered vaccination records determined at step 1110. Step 1120 may also include determining, at step 1130, whether the vaccination subsequence fits the set of rules identified at step 1115.

Step 1135 of method 1100 can include deriving a set of valid vaccination sequences with corresponding vaccination schedules. For example, vaccination subsequences that meet the vaccination schedules are determined to be effective vaccinations.

Step 1140 of method 1100 can include selecting the most relevant set of valid vaccination sequences derived at step 1135. For example, a preferred vaccination sequence can be selected first by a smallest remaining dose count (e.g., where having a value of 0 indicates a fully completed vaccination schedule), then by the most recent last vaccination date. If at step 1140 no matching sequences are found, the method 1100 advances to step 1145 which is indicative of the user having no vaccination records, which may cause the various user interfaces displaying health status check data of the user to indicate the user has no vaccination records. If at step 1140 it is determined that the user has a remaining dose greater than 0 remaining for a particular vaccination sequence, the method 1100 advances to step 1150 which is indicative of the user needing one or more additional doses to complete a vaccination sequence, which may cause the various user interfaces displaying health status check data of the user to indicate the user is not yet fully vaccination, is not yet vaccinated, or is undergoing vaccination. If at step 1140 it is determined that the user has no remaining doses for a particular vaccination sequence, the method 1100 advances to step 1155 which is indicative of the user having effectively completed a vaccination sequence, which may cause the various user interfaces displaying health status check data of the user to indicate the user is fully vaccinated.

Referring to FIG. 12, a graphical user interface 1200 showing the user's profile is shown according to an example embodiment. The graphical user interface 1200 can be displayed via the client application 640 or via a website or stand-alone application (e.g., a website or application hosted or controlled by the provider computing system 200). The graphical user interface 1200 includes an image 1205 of the user, a badge 1210 (e.g., an STI screening badge, an HIV status badge, a prescription badge, a vaccination status badge, a general verification badge such as a badge indicating the user has been verified by the provider computing system 200, etc.), and an icon 1215 that can be interacted with to reveal additional information of the user. In some embodiments, no health information is displayed on user interface 1200 such that the badge 1210 would not be indicative of any particular health status (e.g., the badge 1210 would only be indicative of the user having been verified by the provider computing system 200, such as being verified by or via information received from the identity verification computing system 400). In some embodiments, interacting (e.g., clicking, selecting, tapping, swiping) with any part of the displayed card can cause the card to “flip over” or otherwise reveal additional information by displaying graphical user interface 1300.

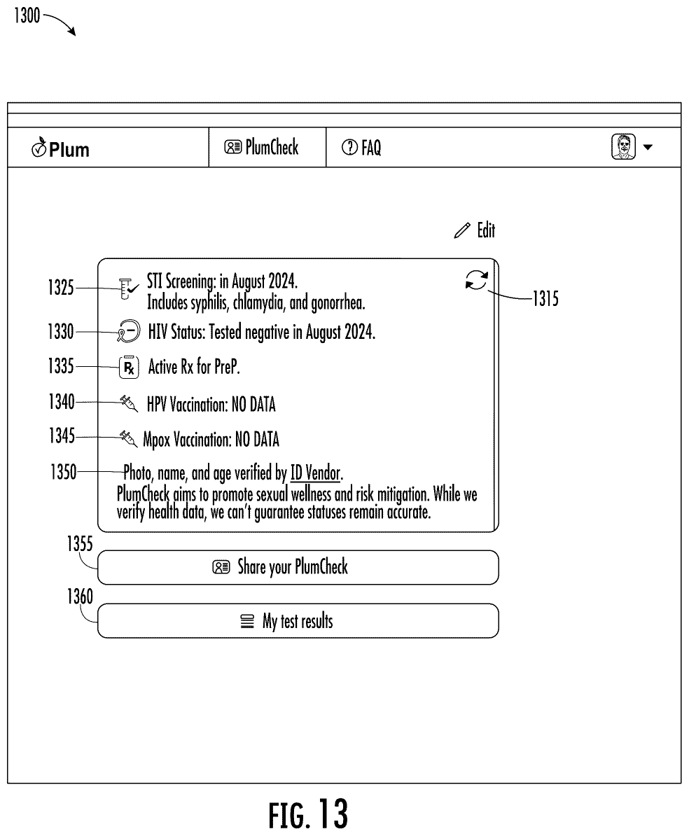

Referring to FIG. 13, another graphical user interface 1300 showing the user's profile is shown according to an example embodiment. The graphical user interface 1300 can be displayed via the client application 640 or via a website or stand-alone application (e.g., a website or application hosted or controlled by the provider computing system 200). The graphical user interface 1300 includes a plurality of user interface elements providing additional information about the user's health statuses and identity. The plurality of user interface elements include an STI screening user interface element 1325 (e.g. a badge or text indicating the last time the user underwent STI screening and infections tested for), an HIV status user interface element 1330 (e.g., indicating whether the user is HIV negative and the last time the user was tested for HIV), an active prescription user interface element 1335 (e.g., indicating an active prescription hat the user has been prescribed, such as PrEP), a first vaccination status user interface element 1340 (e.g., indicating whether the user has received an HPV vaccination and the number of doses and dates of doses), a second vaccination status user interface element 1345 (e.g., indicating whether the user has received a vaccination for Mpox and if so the last time the user received the vaccination), and an identity verification user interface element 1350 (e.g., indicating the user's photo, name, and age were verified, for example, in some cases by a third party such as the identity verification computing system 400). In some embodiments, the graphical user interface 1200 and the graphical user interface 1300 may be combined, such that all or a subset of the information shown on the graphical user interface 1200 and the graphical user interface 1300 can be shown together on a same graphical user interface.

The graphical user interface 1300 also includes a share user interface element 1355 that enables the user to share their verified information with another user. For example, the share user interface element 1355 can be configured to enable the user to send a link or QR code to another user via text message, email, or chat. The graphical user interface 1300 also includes a test results user interface element 1360 configured to enable the user to view test result information that the provider computing system 200 received from the medical record provider computing systems 300. The graphical user interface 1300 also includes an icon 1315 that can be interacted with to reveal the previous user interface (e.g., graphical user interface 1200). In some embodiments, interacting (e.g., clicking, selecting, tapping, swiping) with any part of the displayed card can cause the card to “flip over” or otherwise reveal the prior user interface.

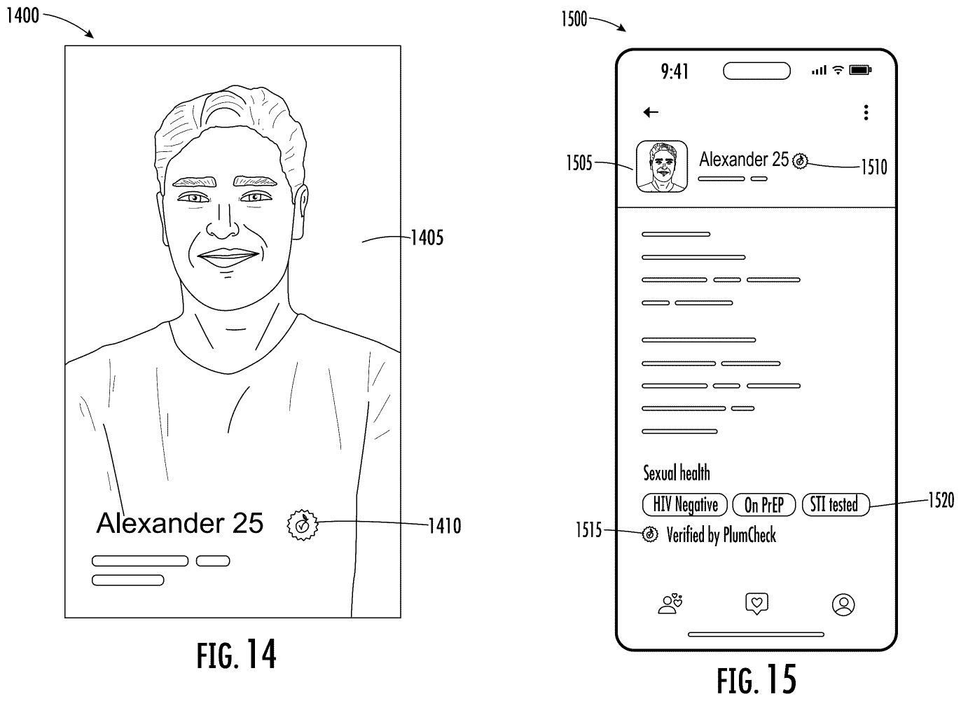

Referring to FIG. 14, another graphical user interface 1400 showing the user's profile is shown according to an example embodiment. The graphical user interface 1400 can be displayed via the client application 640, via a website (e.g., a website hosted or controlled by the provider computing system 200), or via a user profile associated with a platform computing system 500. The graphical user interface 1400 includes an image 1405 of the user and a badge 1410 (e.g., a sexual wellness check badge, an HIV status badge, a prescription badge, a vaccination status badge, a general verification badge such as a badge indicating the user has been verified by the provider computing system 200, etc.). In some embodiments, no health information is displayed on user interface 1400 such that the badge 1410 would not be indicative of any particular health status (e.g., the badge 1410 would only be indicative of the user having been verified by the provider computing system 200, such as being verified by or via information received from the identity verification computing system 400). In some embodiments, interacting (e.g., clicking, selecting, tapping, swiping) with any part of the displayed image, or by interacting with the badge 1410, can cause additional information of the user to be revealed, such as by displaying graphical user interface 1500.

Referring to FIG. 15, another graphical user interface 1500 showing the user's profile is shown according to an example embodiment. The graphical user interface 1500 can be displayed via the client application 640, via a website (e.g., a website hosted or controlled by the provider computing system 200), or via a user profile associated with a platform computing system 500. The graphical user interface 1500 includes an image 1505 of the user, a badge 1510 (e.g., a sexual wellness check badge, an HIV status badge, a prescription badge, a vaccination status badge, a general verification badge such as a badge indicating the user has been verified by the provider computing system 200, etc.), a verification user interface element 1515 (e.g., indicating the user's information has been verified by the provider computing system 200), and additional health user interface elements 1520 (e.g., an HIV Negative verification, a current prescription verification, and an STI test verification). In some embodiments, no health information is displayed on user interface 1500 such that the badge 1510 would not be indicative of any particular health status (e.g., the badge 1510 would only be indicative of the user having been verified by the provider computing system 200, such as being verified by or via information received from the identity verification computing system 400).

Referring to FIG. 16, a graphical user interface 1600 showing the user's profile for limited time period is shown according to an example embodiment. The graphical user interface 1600 can be displayed via the client application 640, via a website (e.g., a website hosted or controlled by the provider computing system 200), or via a user profile associated with a platform computing system 500. The graphical user interface 1600 includes an image 1605 of the user, a badge 1610 (e.g., a sexual wellness check badge, an HIV status badge, a prescription badge, a vaccination status badge, a general verification badge such as a badge indicating the user has been verified by the provider computing system 200, etc.), an icon 1615 that can be interacted with to reveal additional information of the user, and a countdown timer 1620 indicating how much time remaining the viewer has to view the graphical user interface 1600. In some embodiments, no health information is displayed on user interface 1600 such that the badge 1610 would not be indicative of any particular health status (e.g., the badge 1610 would only be indicative of the user having been verified by the provider computing system 200, such as being verified by or via information received from the identity verification computing system 400). In some embodiments, interacting (e.g., clicking, selecting, tapping, swiping) with any part of the displayed card can cause the card to “flip over” or otherwise reveal additional information by displaying graphical user interface 1700. In some embodiments, the user can send a link to another user directing the other user to graphical user interface 1600, which can be configured to be displayed to the user only for a set time period (e.g., 1 minute, 2 minutes, 3 minutes, 5 minutes, 1 hour, 2 hours, etc.).

Referring to FIG. 17, another graphical user interface 1700 showing the user's profile for limited time period is shown according to an example embodiment. The graphical user interface 1700 can be displayed via the client application 640, via a website (e.g., a website hosted or controlled by the provider computing system 200), or via a user profile associated with a platform computing system 500. The graphical user interface 1700 includes an icon 1715 that can be interacted with to reveal the previous user interface (e.g., graphical user interface 1600). In some embodiments, interacting (e.g., clicking, selecting, tapping, swiping) with any part of the displayed card can cause the card to “flip over” or otherwise reveal the prior user interface. The graphical user interface 1700 also includes a countdown timer 1720 indicating how much time remaining the viewer has to view the graphical user interface 1700.

The graphical user interface 1700 also includes a plurality of user interface elements providing additional information about the user's health statuses and identity. The plurality of user interface elements include an STI screening user interface element 1725 (e.g. a badge or text indicating the last time the user underwent STI screening and infections tested for), an HIV status user interface element 1730 (e.g., indicating whether the user is HIV negative and the last time the user was tested for HIV), an active prescription user interface element 1735 (e.g., indicating an active prescription hat the user has been prescribed, such as PrEP), a first vaccination status user interface element 1740 (e.g., indicating whether the user has received an HPV vaccination and if so the last time the user received the vaccination), a second vaccination status user interface element 1745 (e.g., indicating whether the user has received a vaccination for Mpox and if so the last time the user received the vaccination), and an identity verification user interface element 1750 (e.g., indicating the user's photo, name, and age were verified, for example, in some cases by a third party such as the identity verification computing system 400).

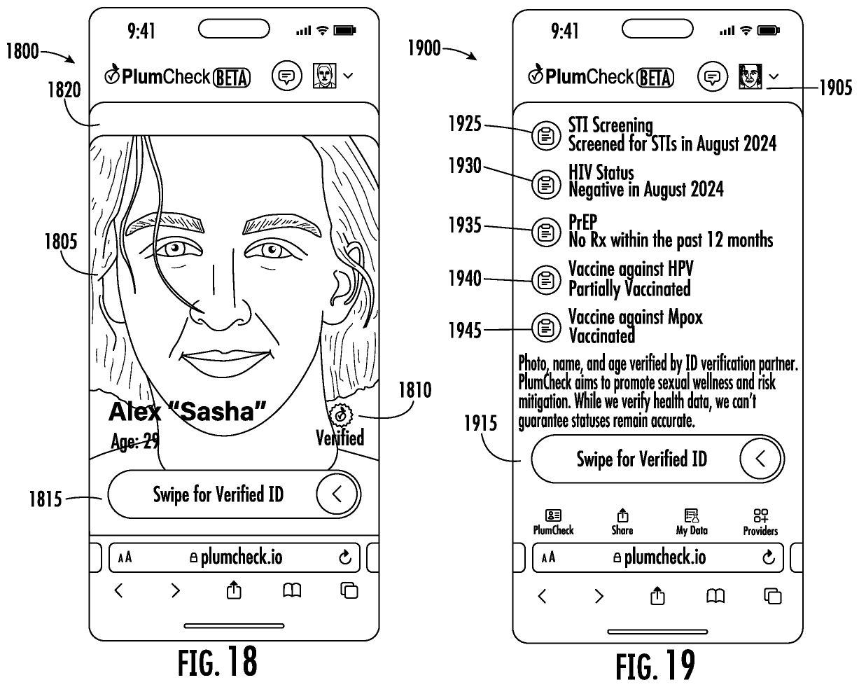

Referring to FIG. 18, another graphical user interface 1800 showing the user's profile is shown according to an example embodiment. The graphical user interface 1800 can be displayed via the client application 640, via a website (e.g., a website hosted or controlled by the provider computing system 200), or via a user profile associated with a platform computing system 500. In one example, the specific graphical user interface 1800 is an account owner (e.g., the user) perspective user interface that can be displayed on an instance of the client application 640 on the user device 600. The graphical user interface 1800 includes an image 1805 of the user and a badge 1810 (e.g., a sexual wellness check badge, an HIV status badge, a prescription badge, a vaccination status badge, a general verification badge such as a badge indicating the user has been verified by the provider computing system 200, etc.). In some embodiments, no health information is displayed on user interface 1800 such that the badge 1810 would not be indicative of any particular health status (e.g., the badge 1810 would only be indicative of the user having been verified by the provider computing system 200, such as being verified by or via information received from the identity verification computing system 400). In some embodiments, interacting (e.g., clicking, selecting, tapping, swiping) with any part of the displayed image, or by interacting with the badge 1810, can cause additional information of the user to be revealed, such as by displaying graphical user interface 1900. The graphical user interface 1800 also includes an icon 1815 that can be interacted with to reveal additional information of the user, such as displaying graphical user interface 1900.

Referring to FIG. 19, another graphical user interface 1900 showing the user's profile is shown according to an example embodiment. The graphical user interface 1900 can be displayed via the client application 640, via a website (e.g., a website hosted or controlled by the provider computing system 200), or via a user profile associated with a platform computing system 500. In one example, the specific graphical user interface 1900 is an account owner (e.g., the user) perspective user interface that can be displayed on an instance of the client application 640 on the user device 600. The graphical user interface 1900 includes an icon 1915 that can be interacted with to reveal the previous user interface (e.g., graphical user interface 1800).

The graphical user interface 1900 also includes a plurality of user interface elements providing additional information about the user's health statuses and identity. The plurality of user interface elements include an STI screening user interface element 1925 (e.g. a badge or text indicating the last time the user underwent STI screening and infections tested for), an HIV status user interface element 1930 (e.g., indicating whether the user is HIV negative and the last time the user was tested for HIV), an active prescription user interface element 1935 (e.g., indicating an active prescription hat the user has been prescribed, such as PrEP), a first vaccination status user interface element 1940 (e.g., indicating whether the user has received an HPV vaccination and if so the last time the user received the vaccination), a second vaccination status user interface element 1945 (e.g., indicating whether the user has received a vaccination for Mpox and if so the last time the user received the vaccination), and in some embodiments, an identity verification user interface element (e.g., indicating the user's photo, name, and age were verified, for example, in some cases by a third party such as the identity verification computing system 400).

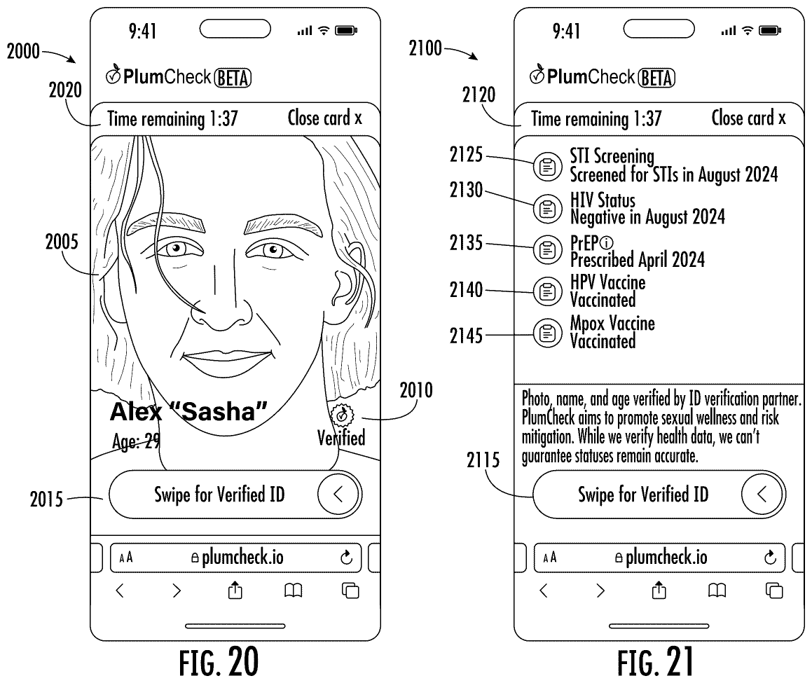

Referring to FIG. 20, another graphical user interface 2000 showing the user's profile for limited time period is shown according to an example embodiment. The graphical user interface 2000 can be displayed via the client application 640, via a website (e.g., a website hosted or controlled by the provider computing system 200), or via a user profile associated with a platform computing system 500. In one example, the specific graphical user interface 2000 is a third-party perspective user interface that can be displayed on an instance of the client application 640 on the third-party user device 600. The graphical user interface 2000 includes an image 2005 of the user and a badge 2010 (e.g., a sexual wellness check badge, an HIV status badge, a prescription badge, a vaccination status badge, a general verification badge such as a badge indicating the user has been verified by the provider computing system 200, etc.), and a countdown timer 2020 indicating how much time remaining the user has to view the graphical user interface 2000. In some embodiments, no health information is displayed on user interface 1900 such that the badge 1910 would not be indicative of any particular health status (e.g., the badge 1910 would only be indicative of the user having been verified by the provider computing system 200, such as being verified by or via information received from the identity verification computing system 400). In some embodiments, interacting (e.g., clicking, selecting, tapping, swiping) with any part of the displayed image, or by interacting with the badge 2010, can cause additional information of the user to be revealed, such as by displaying graphical user interface 2100. The graphical user interface 2000 also includes an icon 2015 that can be interacted with to reveal additional information of the user, such as displaying graphical user interface 2100.

Referring to FIG. 21, another graphical user interface 2100 showing the user's profile for limited time period is shown according to an example embodiment. The graphical user interface 2100 can be displayed via the client application 640, via a website (e.g., a website hosted or controlled by the provider computing system 200), or via a user profile associated with a platform computing system 500. In one example, the specific graphical user interface 2100 is a third-party perspective user interface that can be displayed on an instance of the client application 640 on the third-party user device 600. The graphical user interface 2100 includes an icon 2115 that can be interacted with to reveal the previous user interface (e.g., graphical user interface 2000), and a countdown timer 2120 indicating how much time remaining the user has to view the graphical user interface 2100.

The graphical user interface 2100 also includes a plurality of user interface elements providing additional information about the user's health statuses and identity. The plurality of user interface elements include an STI screening user interface element 2125 (e.g. a badge or text indicating the last time the user underwent STI screening and infections tested for), an HIV status user interface element 2130 (e.g., indicating whether the user is HIV negative and the last time the user was tested for HIV), an active prescription user interface element 2135 (e.g., indicating an active prescription hat the user has been prescribed, such as PrEP), a first vaccination status user interface element 2140 (e.g., indicating whether the user has received an HPV vaccination and if so the last time the user received the vaccination), a second vaccination status user interface element 2145 (e.g., indicating whether the user has received a vaccination for Mpox and if so the last time the user received the vaccination), and in some embodiments, an identity verification user interface element (e.g., indicating the user's photo, name, and age were verified, for example, in some cases by a third party such as the identity verification computing system 400).

It should be noted while certain parts of this application, including references to FIGS. 8-10 and 12-21, refer to example use cases relating a dating application and the sharing of health status check information relating to sexual health, it will be appreciated that such use cases are provided for example only and in no way are limiting. As such, it will be appreciated that the concepts disclosed herein could be applicable to any number of use cases where the sharing of health information would be beneficial, including the other non-sexual health and dating application use cases disclosed herein.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be configured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components configured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. A provider computing system, comprising:
a processing circuit configured to:
receive personal data of a user;
create a user account associated with the provider computing system for the user based on the personal data of the user;
receive, via a first application programming interface (API), identity verification information of the user from an identity verification computing system;
provide, via a second API, a request for health data of the user to a medical record provider computing system;
receive, via the second API, health data of the user from the medical record provider computing system;
determine a health status of the user based on the health data; and
cause the health status of the user to be displayed on at least one of a user device associated with the user via a first instance of a client application, a third-party user device of a third party via a second instance of the client application, or via a platform computing system.

2. The provider computing system of claim 1, wherein the processing circuit is configured to receive a preference of the user, wherein the preference identifies a specific type of health data for verification.

3. The provider computing system of claim 2, wherein the request for health data of the user is based on the preference, and wherein the received health data of the user includes health data relating to the specific type of health data.

4. The provider computing system of claim 1, wherein the health data received via the second API comprises at least one of one or more medical codes or related values.

5. The provider computing system of claim 4, wherein determining a health status of the user based on the health data comprises interpreting the one or more medical codes to determine a health status of the user.

6. The provider computing system of claim 5, wherein the processing circuit is further configured to determine a display code associated with the health status of the user, wherein the display code is associated with at least one of a graphical or textual user interface element indicating the health status, and wherein the display code is stored in a memory.

7. The provider computing system of claim 6, wherein the processing circuit is further configured to destroy the health data of the user after storing the display code in the memory.

8. The provider computing system of claim 7, wherein the processing circuit is configured to receive, via the second API, updated health data of the user.

9. The provider computing system of claim 8, wherein the processing circuit is configured to determine an updated health status of the user based on the updated health data, determine an updated display code associated with the updated health status of the user, and replace the display code with the updated display code in the memory.

10. The provider computing system of claim 1, wherein the health status is displayed as one or more badges affixed to a user profile of the user that is viewable by the third-party via one or more of the user device, the third-party user device, or the platform computing system.

11. The provider computing system of claim 1, wherein the processing circuit is configured to use the identity verification information obtained via the first API to confirm ownership of the health data obtained via the second API.

12. A provider computing system, comprising:
a processing circuit configured to:
receive personal data of a user;
provide, via an application programming interface (API), a request for health data of the user to a medical record provider computing system;
receive, via the API, health data of the user from the medical record provider computing system;
determine a health status of the user based on the health data; and
cause the health status of the user to be displayed on at least one of a user device associated with the user via a first instance of a client application, a third-party user device of a third party via a second instance of the client application, or via a platform computing system.

13. The provider computing system of claim 12, wherein the health data received via the API comprises at least one of one or more medical codes or related values.

14. The provider computing system of claim 13, wherein determining a health status of the user based on the health data comprises interpreting the one or more medical codes to determine a health status of the user.

15. The provider computing system of claim 14, wherein the processing circuit is further configured to determine a display code associated with the health status of the user, wherein the display code is associated with at least one of a graphical or textual user interface element indicating the health status, and wherein the display code is stored in a memory.

16. The provider computing system of claim 12, wherein the health status is displayed as one or more badges affixed to a user profile of the user that is viewable by the third-party via at least one of the user device, the third-party user device, or the platform computing system, and the displayed health status of the user includes a date associated with the health status.

17. The provider computing system of claim 12, wherein the platform computing system is a social media platform computing system.

18. A method comprising:
receiving, by a processing circuit of a provider computing system, personal data of a user;
providing, by the processing circuit and via an application programming interface (API), a request for health data of the user to a medical record provider computing system;
receiving, by the processing circuit and via the API, health data of the user from the medical record provider computing system;
determining, by the processing circuit, a health status of the user based on the health data; and
causing, by the processing circuit, the health status of the user to be displayed on at least one of a user device associated with the user via a first instance of a client application, a third-party user device of a third party via a second instance of the client application, or via a platform computing system.

19. The method of claim 18, wherein the health data received via the API comprises at least one of one or more medical codes or related values, and determining a health status of the user based on the health data comprises interpreting the one or more medical codes to determine a health status of the user.

20. The method of claim 18, further comprising determining a display code associated with the health status of the user, wherein the display code is associated with at least one of a graphical or textual user interface element indicating the health status.
